(19) United States
(12) Patent Application Publication
(10) Pub. No.: US 2026/0176565 A1
(43) Pub. Date: Jun. 25, 2026

(54) SYSTEMS FOR MIXING, GAS EXCHANGE AND ENVIRONMENTAL REGULATION IN BIOREACTORS

(71) Applicant: VANDERBILT UNIVERSITY, Nashville, TN (US)

(72) Inventors: Dmitry Markov, Nashville, TN (US); Ronald S. Reiserer, Nashville, TN (US); Eric C. Spivey, Nashville, TN (US); John P. Wikswo, Brentwood, TN (US); David K. Schaffer, Nashville, TN (US)

(21) Appl. No.: 19/426,304

(22) Filed: Dec. 19, 2025

Related U.S. Application Data
(60) Provisional application No. 63/736,274, filed on Dec. 19, 2024.

(51) Int. Cl. C12M 1/00; C12M 1/32; C12M 1/34

(52) U.S. Cl. CPC C12M 29/04; C12M 23/12; C12M 23/58; C12M 29/10; C12M 41/12; C12M 41/26; C12M 41/32; C12M 41/34

(57) ABSTRACT

Systems and methods are provided for mixing and gas exchange in bioreactors, particularly small-volume and high-aspect-ratio bioreactors such as wells of multi-well plates. A movable displacement body is positioned within a vessel containing a liquid culture medium and is periodically moved relative to the vessel to displace and mix the medium. The displacement body includes or is associated with a gas exchange interface that permits transfer of gases between the gas exchange interface and the liquid culture medium during movement of the displacement body. Mixing and gas exchange occur concurrently as a result of displacement-based flow, enabling efficient oxygenation, carbon dioxide removal, and environmental control while limiting shear stress and avoiding gas sparging.

### CROSS-REFERENCE TO RELATED PATENT APPLICATION

This application claims priority to and the benefit of U.S. Provisional Patent Application Ser. No. 63/736,274, filed Dec. 19, 2024, which is incorporated herein by reference in its entirety.

### STATEMENT AS TO RIGHTS UNDER FEDERALLY-SPONSORED RESEARCH

This invention was made with government support under Grant No. 2117782 awarded by the National Science Foundation, and Grant No. EB030410 awarded by the National Institutes of Health (NIH) National Institute of Biomedical Imaging and Bioengineering (NIBIB). The government has certain rights in the invention.

### FIELD OF THE INVENTION

This invention relates generally to bioreactors and cell culture systems, and more particularly to systems and methods for mixing, oxygenation, carbon dioxide control, and environmental regulation in small-scale and parallel bioreactors.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Bioreactors are widely used for culturing microbial, plant, and animal cells for applications including biopharmaceutical manufacturing, cell and gene therapy, tissue engineering, metabolic studies, and high-throughput screening. Effective operation of a bioreactor requires controlled delivery of nutrients and gases, removal of metabolic byproducts, maintenance of uniform environmental conditions, and minimization of mechanical stress on cells.

At large scales, mixing and gas exchange are typically achieved using mechanical impellers, sparging, airlift circulation, or external recirculation loops. At smaller scales, including milliliter-scale bioreactors and deep wells of multi-well plates, such approaches become problematic. Rotational stir bars or miniature impellers often generate excessive shear, create vortices that interfere with sampling, or fail to provide adequate vertical mixing. Gas sparging can lead to foaming, bubble-induced cell degradation, and the need for antifoaming agents that alter cell physiology.

Headspace-only gas exchange is frequently inadequate in deep or high-aspect-ratio vessels due to limited surface-to-volume ratio. Static gas-permeable membranes or hollow fibers can improve gas transfer but are prone to localized supersaturation, bubble nucleation, fouling, and incomplete mixing unless high flow velocities are used.

Therefore, there exists a need for improved bioreactor systems that provide efficient mixing and gas exchange at small scales while minimizing shear stress, foaming, and system complexity, and that are compatible with parallelized and automated operation.

### SUMMARY OF THE INVENTION

This invention addresses the foregoing needs by providing systems and methods in which mixing and gas exchange are achieved through displacement-based motion of a movable body within a bioreactor vessel. The displacement member is periodically moved relative to the vessel, causing the liquid culture medium in the bioreactor vessel to be displaced through confined regions and bulk volumes, thereby mixing the medium.

In one aspect of the invention, the bioreactor system comprises a bioreactor vessel configured to contain a culture medium; and a displacement membrane gas exchanger and mixer (DM-GEM) configured to be positioned within the vessel, wherein the DM-GEM comprises a displacement member having an outer cross-sectional area smaller than an inner cross-sectional area of the vessel, thereby defining a fluid annulus between the displacement member and a wall of the vessel, wherein the displacement member has a gas-permeable surface defining an interior chamber therewith; and an actuator mechanism coupled to the displacement member and configured to vertically oscillate the displacement member through a substantial portion of the culture medium in the vessel. The vertical oscillation of the displacement member displaces the culture medium through the fluid annulus between the displacement member and the vessel wall to mix the medium while simultaneously exchanging gas across the gas-permeable wall of the displacement member.

In one embodiment, the gas-permeable surface of the displacement member is configured to permit diffusion of oxygen from the interior chamber into the culture medium and diffusion of carbon dioxide from the culture medium into the interior chamber.

In one embodiment, the displacement member is an extensible bulb formed of an elastomer, and a diameter of the extensible bulb varies in response to gas pressure within the interior chamber.

In one embodiment, the vertical oscillation of the displacement member is configured to force the culture medium to flow through the annulus between the displacement member and the vessel wall; generate laminar flow within the annulus to minimize shear forces on suspended cells; and generate vortices and turbulent mixing in the wake region above and below the displacement member to promote large-scale mixing throughout the culture medium.

In one embodiment, the actuator mechanism is an electromagnetic actuator comprising a solenoid and at least one permanent magnet coupled to the displacement member, wherein current in the solenoid is controlled to generate the vertical oscillation of the displacement member.

In one embodiment, gas pressure within the interior chamber is controlled to regulate at least one of a gas transfer rate, a mixing intensity, and a shear rate experienced by cells.

In one embodiment, the displacement member is oscillated with a waveform selected to control a shear rate and mixing within the culture medium.

In one embodiment, gas transfer is bidirectional, permitting delivery of oxygen into the medium and removal of carbon dioxide from the medium. Gas composition, pressure, flow rate, and humidity within the displacement member may be actively controlled to regulate dissolved gas concentrations and pH. Mixing intensity and shear exposure may be tuned by adjusting displacement amplitude, frequency, velocity, or effective cross-sectional area of the displacement body.

In one embodiment, the system further comprises a gas management unit configured to deliver a gas mixture into the culture medium via diffusion and/or convection across the gas-permeable wall of the displacement member; receive metabolic gases from the culture medium via diffusion into the interior chamber of the displacement member; and/or dynamically alter dimensions of the displacement member to adjust a width of the fluid annulus and tune a mixing rate.

In one embodiment, the system further comprises a gas supply fluidly coupled to the interior chamber of the displacement member to deliver the gas mixture with a controlled gas composition; a headspace coupled to the vessel and configured to control gas partial pressures and flow rates in a headspace of the bioreactor vessel for additional gas exchange; and/or a humidification unit to humidify gas delivered to both the displacement member and the headspace to prevent evaporation of the culture medium.

In one embodiment, the DM-GEM further functions for temperature control, by regulating the temperature and flow rate of the gas delivered to the interior chamber of the displacement member to control the temperature of the culture medium.

In one embodiment, the DM-GEM is implemented as a cartridge configured for insertion into a well of a multi-well plate, wherein the cartridge further comprises coaxial conduits including a fixed inner tube delivering gas to the interior chamber and a movable outer tube removing gas from the interior chamber and controlling the vertical position of the displacement member.

In one embodiment, the bioreactor vessel is one well of a multi-well plate, and the system further comprises a plurality of displacement members operating in parallel in respective wells.

In one embodiment, the system further comprises one or more sensors configured to measure at least one of pH, temperature, dissolved oxygen, carbon dioxide, lactate, optical density, and metabolite concentration in the culture medium.

In one embodiment, the system is further configured for perfusion operation, including a means for continuous or intermittent removal of spent media and replacement with fresh media while retaining suspended cells in the bioreactor vessel.

In another aspect of the invention, the bioreactor system comprises a bioreactor vessel configured to contain a culture medium; a gas-permeable member dimensioned to be moved vertically within the vessel to define a fluid annulus between the gas-permeable member and a wall of the vessel; and a drive mechanism operably coupled to the gas-permeable member, and configured to cycle the vertical position of the gas-permeable member to displace the culture medium and promote mixing while simultaneously facilitating gas diffusion and/or convection across a surface of the gas-permeable member.

In one embodiment, the gas-permeable member is an elastomeric bulb.

In one embodiment, the system further comprises a means for adjusting the internal pressure of the gas-permeable member to modify dimensions of the gas-permeable member, thereby controlling a width of the annulus and dynamically tuning a rate of displacement mixing.

In one embodiment, the vertical movement of the gas-permeable member is configured to generate laminar fluid flow through the annulus to minimize shear forces on cultured cells in the culture medium; and/or optimize turbulent fluid flow (vortices) in bulk culture medium upstream and downstream of the gas-permeable member to promote homogenization.

In one embodiment, the system further comprises a gas management system configured to flow a gas mixture through an interior of the gas-permeable member to enable bidirectional gas exchange, including delivery of oxygen and removal of carbon dioxide.

In one embodiment, the drive mechanism comprises a mechanical crank, a pneumatic piston, a pneumatic bellows, and/or an electromagnetic actuator.

In yet another aspect of the invention, the bioreactor system comprises a vessel configured to contain a culture medium; a movable displacement body positioned at least partially within the vessel; an actuator configured to move the displacement body reciprocally along a substantially vertical path within the vessel so as to displace at least a substantial portion of the culture medium and thereby mix the culture medium; and a gas exchange interface integral to or associated with the displacement body and configured to facilitate exchange of at least one gas between the gas exchange interface and the culture medium in the vessel during the movement of the displacement body, wherein mixing of the culture medium and gas exchange occur concurrently as a result of movement of the displacement body within the vessel.

In one embodiment, the displacement body includes or is associated with the gas exchange interface, such as a gas-permeable wall or membrane, that permits transfer of gases between a gas source and the liquid culture medium during movement of the displacement body. As the displacement body traverses the vessel, substantially all portions of the liquid culture medium are periodically brought into proximity with the gas exchange interface, enhancing gas transfer efficiency and homogeneity.

In one embodiment, the movable displacement body has a cross-sectional shape comprising a spherical, spheroidal, cylindrical, or polygonal shape.

In one embodiment, the gas exchange interface comprises a gas-permeable portion of the displacement body.

In one embodiment, the gas exchange interface is configured to permit transfer of oxygen into the culture medium and transfer of carbon dioxide out of the culture medium.

In one embodiment, the movement of the displacement body causes liquid flow through a confined flow region between the displacement body and a wall of the vessel.

In one embodiment, the confined flow region produces a first flow regime adjacent to the displacement body and a second flow regime remote from the displacement body, the first and second flow regimes being different.

In one embodiment, the movement of the displacement body mixes the culture medium while limiting shear stress experienced by cells suspended in the medium.

In one embodiment, the location of the displacement body over time can be specified by a particular periodic waveform, such as sinusoidal, ramps with different up and down rates, or waveforms with intervals with no vertical motion.

In one embodiment, at least one of displacement amplitude, displacement frequency, displacement velocity, or effective displacement cross-section of the displacement body is adjustable during operation.

In one embodiment, gas transfer across the gas exchange interface is regulated by controlling gas pressure, gas composition, or gas flow rate.

In one embodiment, the displacement body is deformable such that an effective cross-sectional dimension of the displacement body varies during operation.

In one embodiment, the actuator comprises at least one of an electromagnetic actuator, a pneumatic actuator, a hydraulic actuator, a mechanical linkage, and a motor-driven actuator.

In one embodiment, the displacement body and actuator are integrated into a removable cartridge configured to be inserted into the vessel.

In one embodiment, the system further comprises a plurality of vessels each having a respective displacement body, enabling parallel operation of multiple bioreactors.

In one embodiment, the system further comprises control of at least one of dissolved oxygen, carbon dioxide and lactate concentration, pH, and temperature of the liquid culture medium, and/or the humidity in the headspace or the displacement member.

In one embodiment, the system further comprises one or more sensors configured to monitor at least one parameter of the liquid culture medium or gas exchanged through the gas exchange interface.

The systems may be implemented as compact cartridges insertable into individual wells of a multi-well plate, enabling massively parallel operation. The invention is compatible with batch, fed-batch, and continuous or intermittent perfusion modes, and may incorporate sensors and control systems for automated operation.

In one aspect, the invention relates to a system for parallel bioreactor operation, comprising a well plate comprising a plurality of wells; a plurality of cartridge units, each cartridge unit corresponding to one of the plurality of wells and comprising a movable displacement body, an actuator configured to impart reciprocal motion to the displacement body within its corresponding well, and a gas exchange interface integral to or associated with the displacement body; and a controller operably coupled to the plurality of cartridge units and configured to independently or synchronously control movement of each displacement body and gas delivery to each gas exchange interface.

In one embodiment, each cartridge unit further comprises at least one integrated sensor for monitoring a condition within its corresponding well.

In one embodiment, the actuators for the plurality of cartridge units are configured to move in unison, oscillating a corresponding array of displacement bodies relative to the well plate.

In one embodiment, the system further comprises a gas management unit configured to supply a controllable gas mixture to the gas exchange interface of each displacement body.

In one embodiment, the gas management unit is further configured to humidify the controllable gas mixture.

In one embodiment, the system further comprises a cell separation means that enables continuous or intermittent perfusion culture with partial removal of condition media and its replacement with fresh media while minimizing cell loss.

In another aspect, the invention relates to a method for culturing cells within a bioreactor vessel containing a culture medium, comprising a gas-permeable displacement member within the culture medium, wherein the displacement member has a gas-permeable surface defining an interior chamber therewith; oscillating the displacement member vertically relative to a wall of the vessel to impart movement to the culture medium; and exchanging gases between the interior chamber of the displacement member and the culture medium across the gas-permeable surface of the displacement member during the oscillation.

In one embodiment, the bioreactor vessel is a deep well of a multi-well plate.

In one embodiment, the oscillating and exchanging steps are performed to provide adequate vertical mixing and gas transfer despite the small surface-to-volume ratio of the well.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows schematically a displacement membrane gas exchanger and mixer operating in a bioreactor that is implemented in the deep wells of a well plate.

FIG. 2 shows schematically a comparison of CHO growth rates in a shaker flask and a deep well mixed by a 7×2 mm magnetic stir bar operating at speeds of 120 RPM and >300 RPM.

FIG. 3 shows schematically a vertically moving displacer that forces media through an annulus between the moving displacer and the fixed wall of the bioreactor vessel, creating a combination of laminar flow in the annulus and vortices in the bulk culture media that mix the cells and media in a single stroke. Multiple cyclic strokes of the displacer improve the mixing.

FIG. 4 shows schematically fluid velocities in a displacement mixer.

FIG. 5 shows schematically calculated and measured fluid flow from a sphere moving in a fluid-filled tube.

FIG. 6 shows schematically the different hydrodynamic regions in the vicinity of a downward moving displacement mixer.

FIG. 7 shows schematically bidirectional gas exchange across the wall of a gas-exchange bulb in a moving displacement mixer operating in a single well of a deep well plate.

FIG. 8 shows schematically possible mechanical, pneumatic, or hydraulic means to oscillate the vertical position of the bulb of a displacement gas exchanger and mixer.

FIG. 9 shows schematically tests of oscillating PDMS bulb displacement mixing and gas exchange during CHO cell culture, showing both cell count and cell viability.

FIG. 10 shows schematically one embodiment of an electromagnetic drive for the displacement membrane gas exchanger and mixer.

FIG. 11 shows schematically a comparison of CHO cell growth rates in a shaken flask as compared to an electromagnetically actuated displacement membrane gas exchanger and mixer.

FIG. 12 shows schematically various waveforms that would provide different time-varying control of the mixer position within the well, showing position plotted as a function of time.

FIG. 13 shows schematically the time dependence of the readout of a fluorescent oxygen sensor in the liquid above a moving PDMS displacement mixer bulb filled with 1 psi air, beginning with 22° C. water that was initially saturated with nitrogen.

FIG. 14 shows schematically a determination of the oxygen delivery rate using the observed time dependence of oxygen concentration in a 2 mL bioreactor, starting with deoxygenated water at 22° C.

FIG. 15. shows schematically measurements of oxygen delivered into a 2 mL bioreactor using a displacement membrane gas exchanger and mixer operating at 0.5 and 1.5 psi O₂ pressure, as compared to the direct bubbling of O₂ into the media.

FIG. 16 shows schematically an example of the quantitative analysis of oxygen delivery data to determine the k_{L}a for both displacement membrane gas exchanger and mixer delivery at two pressures and direct bubbling into water. The slope of each line is the volumetric oxygen transfer coefficient, k_{L}a.

FIG. 17 shows schematically a demonstration of how 5% CO₂/air delivered into unbuffered water by the displacement membrane gas exchanger and mixer bulb can be used to control media pH.

FIG. 18 shows schematically an embodiment of the invention with twelve displacement membrane gas exchanger and mixer units operating in a well plate.

FIG. 19 shows schematically alternative embodiments wherein an array of displacement membrane gas exchanger and mixer bulbs is moved relative to the well plate by either periodically raising and lowering the array, or periodically raising and lowering the well plate.

FIG. 20 shows schematically a vertical electromagnetic actuator cartridge for a displacement membrane gas exchanger and mixer using concentric gas delivery and withdrawal tubes and no moving, flexible interconnecting tubing.

FIG. 21 shows schematically the use of the concentric combination of an oscillating outer tube that both controls the oscillating vertical position of a gas exchanger/mixer bulb and removes gas from the interior of the bulb, and a fixed inner tube that delivers gas to the interior of the bulb.

FIG. 22 shows a schematic representation of an array of cartridges that each serve as a complete displacement membrane gas exchanger and mixer for sensing and control of bioreactors implemented in the wells of a well plate.

FIG. 23 shows schematically design details of a displacement membrane gas exchanger and mixer cartridge that can be inserted into any well of a deep 48-well plate.

FIG. 24 shows schematically external and internal details of one embodiment with eight displacement membrane gas exchanger and mixer cartridges that are inserted in a single row of a deep 48-well plate.

FIG. 25. shows schematically various configurations, surfaces, and shapes for displacement gas exchanger and mixer bulbs and the bioreactor vessels in which they operate.

FIG. 26 shows schematically three bulb designs with an adjustable cross-sectional area.

FIG. 27 shows schematically a demonstration that in batch-fed culture, a displacement membrane gas exchanger and mixer can grow CHO cells and achieve live cell densities comparable to or even better than a shaken flask, but that with both methods, the live cell density drops at Day 5 as nutrients are depleted and toxic metabolites accumulate.

FIG. 28 shows schematically operation of a displacement membrane gas exchanger and mixer in two possible modes that support either continuous or intermittent replacement of cell culture media while retaining the cells in the bioreactor.

### DETAILED DESCRIPTION OF THE INVENTION

The invention will now be described more fully hereinafter with reference to the accompanying drawings, in which exemplary embodiments of the invention are shown. The invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the invention. As used herein, the singular forms “a”, “an”, and “the” are intended to include the plural forms as well, unless the context clearly indicates otherwise. It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used herein, “around”, “about”, “substantially” or “approximately” shall generally mean within 20 percent, preferably within 10 percent, and more preferably within 5 percent of a given value or range. Numerical quantities given herein are approximate, meaning that the terms “around”, “about”, “substantially” or “approximately” can be inferred if not expressly stated.

As used herein, the terms “comprise” or “comprising”, “include” or “including”, “carry” or “carrying”, “has/have” or “having”, “contain” or “containing”, “involve” or “involving” and the like are to be understood to be open-ended, i.e., to mean including but not limited to.

As used in this invention, the phrase “at least one of A, B, and C” should be construed to mean a logical (A or B or C), using a non-exclusive logical OR. As used herein, the term “and/or” includes any and all combinations of one or more of the associated listed items.

The term “elastomer”, as used in the disclosure, refers to a polymeric material which can be stretched or deformed and return to its original shape without substantial permanent deformation. Elastomers commonly undergo substantially elastic deformations. Useful elastomers include those comprising polymers, copolymers, composite materials or mixtures of polymers and copolymers. Elastomeric layer refers to a layer comprising at least one elastomer. Elastomeric layers may also include dopants and other non-elastomeric materials. Useful elastomers include, but are not limited to, thermoplastic elastomers, styrenic materials, olefinic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. Exemplary elastomers include, but are not limited to, silicon-containing polymers such as polysiloxanes including poly(dimethyl siloxane) (i.e., PDMS and h-PDMS), poly(methyl siloxane), partially alkylated poly(methyl siloxane), poly(alkyl methyl siloxane) and poly(phenyl methyl siloxane), silicon-modified elastomers, thermoplastic elastomers, styrenic materials, olefinic materials, polyolefin, polyurethane thermoplastic elastomers, polyamides, synthetic rubbers, polyisobutylene, poly(styrene-butadiene-styrene), polyurethanes, polychloroprene and silicones. Embodiments of the invention are illustrated in detail hereinafter with reference to

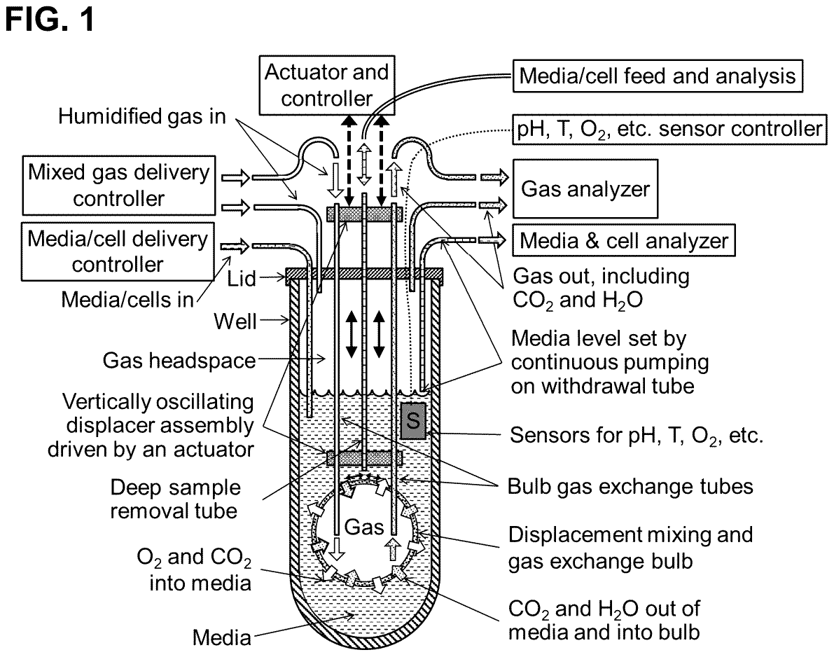

accompanying drawings of FIGS. 1-28. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers or names will be used in the drawings to identify similar elements. It should be understood that one or more steps within a method may be executed in different order (or concurrently) without altering the principles of the invention.

Bioreactors are engineered systems used to culture microbial, animal, or plant cells under controlled and aseptic conditions. They are central to a wide range of applications, including the large-scale production of biopharmaceuticals such as monoclonal antibodies, vaccines, and recombinant proteins; microbial and algal fermentations for fuels, chemicals, and foods; cell and gene therapy manufacturing, involving viral vectors and therapeutic cell expansion; tissue engineering and organ-on-chip systems for modeling physiology and disease; environmental processes such as wastewater treatment and bioremediation; and emerging areas such as cultured meat, precision fermentation of food ingredients, and specialized bioconversions using immobilized cells or enzymes. In general, bioreactors are designed to maintain temperature, pH, nutrient levels, dissolved and headspace gases, and metabolite concentrations within defined limits while providing appropriate mixing, mass transfer, and mechanical forces. Most commercial bioreactors incorporate internal and external sensors and automated control systems to regulate environmental conditions, support cell growth or product formation, and ensure consistent, reproducible operation.

For this invention, we focus on cells grown either in suspension or attached to suspended carrier beads, but this invention is not limited to this particular type of cell culture. This invention is immediately applicable to small bioreactors, which are often used to screen candidate cellular clones for their potential capabilities as a production strain in large bioreactors, and hence it is important to have small bioreactors that can be replicated in large numbers to support massively parallel experiments for either screening large numbers of potential clones, or studying coupled small bioreactors that together simulate the zonation known to exist in large bioreactors. The design of the requisite bioreactors is guided by a number of factors.

Bioreactors are typically sterilized prior to use and are operated aseptically to minimize contamination during the delivery of liquids or gases and the removal of samples. Bioreactors frequently operate at elevated temperatures, and temperature control must account for heat losses to the environment, the temperature of incoming and outgoing gases and liquids, and any heat generated by cellular metabolism. The partial pressures of gases dissolved in the culture medium or present in the headspace above can be critical to proper operation and achieving high product yields.

Depending on the operating mode, it may also be necessary to prevent cells from exiting the bioreactor or to separate cells from the effluent stream and return them to the vessel, which would typically be accomplished with either tangential flow microfiltration, alternating tangential flow filtration, spiral or other kinetic cell separators, or other means.

Several mechanical operations internal to the bioreactor are essential for proper function: addition of nutrients; removal of cells, metabolites, and other biochemical products; gas exchange (e.g., oxygen and carbon dioxide); and mixing or stirring that minimizes gradients in temperature, pH, and chemical concentration while limiting the shear forces and foam generation associated with gas injection, agitation, or fermentation. In large bioreactors, these gradients can become significant enough to create distinct zones within the vessel that differ in temperature, pH, nutrient levels, and dissolved gas concentrations. Transient exposure of a suspended cell to such zones can induce persistent changes in gene expression and reduce overall productivity. This invention focuses primarily on the challenges of gas exchange and mixing.

The density to which cells can be cultured is determined in part by the rate at which nutrients and gases such as oxygen and carbon dioxide can be delivered, by the efficiency with which toxic metabolic products, including dissolved solutes and gases such as carbon dioxide or methane, can be removed, and by the uniformity of various concentrations throughout the bioreactor. Over a volume range that spans at least six or seven orders of magnitude, various bioreactors employ a broad array of mixing technologies, including mechanically driven impellers of various designs, gas-induced circulation in airlift and bubble column systems, wave-generated motion in rocking-bag reactors, orbital shaking of vessels and microtiter plates, jet-or pump-driven recirculation loops, magnetically coupled or levitated stirring devices, rotating wall vessels that create low-shear environments, and microfluidic or diffusion-dominated mixers that rely on structured channel geometries to achieve fluid motion.

For large-volume, industrial-scale bioreactors with working volumes of thousands to tens of thousands of liters, the dominant mixing technologies are mechanical impeller mixing in stirred tank reactors, gas-induced mixing using airlifts or bubble columns, and jet-or pump-driven external circulation. At the medium scale of approximately 100 mL to 1 L, mixing is generally accomplished using mechanical impellers, rocking-induced waves in small single-use bags, orbital shaking in deep-well plates or shake flasks, magnetically coupled impellers, rotating wall bioreactors, and occasionally small airlift reactors. At the smallest scale of approximately 1 mL to 10 mL, mixing can be provided by orbital shaking of well plates, particularly those with non-circular geometries, by perfusion-driven mixing in organ chip devices, microbioreactors, or hollow fiber systems, and by miniature magnetically driven stir bars or levitated impellers. Airlift systems are generally impractical at these very small scales due to excessive foaming (which can complicate fluid level control and compromise the metabolism of cells trapped in foam), and wave motion is ineffective below volumes of roughly 50-100 mL.

Across all bioreactor sizes, from sub-milliliter microfluidic systems to 10,000-liter or larger industrial reactors, gas exchange is achieved through a relatively well-defined set of core technologies, each of which solves the problem in a different hydrodynamic and mass transfer regime, including direct sparging with porous, ring, or fine-bubble diffusers; gas-induced circulation in airlift and bubble column systems; surface aeration and headspace gas control in shaken, rocked, or thin-film configurations; membrane-based oxygenation using hollow fibers, silicone tubing, or flat sheet nanoporous, microporous or otherwise gas-permeable materials; microfluidic and diffusion-dominated gas delivery through polydimethylsiloxane (PDMS) or other permeable structures; jet-or Venturi-based gas entrainment in high-velocity recirculation loops; rotating wall and other low-shear diffusion systems; and, in specialized small-volume applications, oxygen-carrying liquids or chemical oxygen-release materials that supplement or replace direct gas transfer.

For small, well-stirred aerobic bioreactors with modest oxygen demands, gas exchange can often be provided entirely through the headspace, with oxygen diffusing into the liquid at the air-liquid interface and carbon dioxide diffusing out. In miniature bioreactors with large aspect ratios, such as deep wells in multi-well plates, mechanical mixing with a magnetic stir bar is often impractical, because the high rotational speeds required for adequate vertical mixing generate excessive shear and produce deep vortices that interfere with level sensing and sample withdrawal. Although direct oxygen injection through a sparger offers rapid oxygen delivery, the proteins in typical culture media can produce substantial foam, which entrains cells and lifts them out of the culture medium, decreasing environmental uniformity and often requiring defoaming agents that can alter cell physiology and reduce product formation.

With headspace gas exchange in a deep well of a multi-well plate, the surface-to-volume ratio is inversely proportional to the well depth, creating a trade-off between increasing well depth and the total number of viable cells that can be cultured. Increasing the absolute oxygen pressure in the headspace can partially compensate for this limitation, but using high oxygen concentrations at elevated pressure presents technical challenges and clear safety risks. Gas-exchange surface area, and thus the exchange rate, could be increased by using hollow, gas-permeable fibers to deliver oxygen deeper into the medium, as is done in several commercially available perfused hollow fiber bioreactors. However, implementing hollow fiber oxygenators independently in each well of a deep 48-well plate for suspension culture is difficult, particularly because oxygen build-up at the fiber surface can nucleate bubble formation and cause foaming, and because the large number of fibers needed would impede mixing. Unless the media flows across the fibers at a high velocity, the transfer of gas from the fiber into the liquid can be limited by the diffusion rate of the gas in the media; media flowing axially along the outside of the hollow fibers would simply exacerbate the problem of bubble nucleation at the downstream ends of the hollow fibers. The resulting challenge is to design a mechanism that ensures oxygen transferred from the delivery device is rapidly mixed with oxygen-depleted media, thereby minimizing local supersaturation and bubble nucleation at or near the gas exchange surfaces while ensuring a mechanically and chemically homogeneous culture environment.

This invention uses displacement mixing to minimize chemical, thermal, and mechanical gradients in liquids contained in a high-aspect-ratio cylindrical bioreactor, wherein the “mixer” is a smooth object, such as a sphere with a cross-sectional area somewhat smaller than that of the bioreactor vessel, whose position oscillates between the top and bottom of the fluid-filled chamber, displacing liquid upward through the annulus as the mixer descends and downward as it rises. The ratio of the cross-sectional area of the annulus between the mixer and the bioreactor wall to that of the bioreactor itself, together with the oscillation frequency and the viscosity of the medium, determine not only the mixing rate and efficiency but also the mechanical force required to drive mixer displacement. Given the size and the relatively slow movement of the large mixer, the fluid velocities in the annulus will be more uniform than those produced by small, high-speed impellers or stir bars, thereby minimizing the shear forces encountered by the suspended cells. The mechanical simplicity of displacing a mixer over large vertical distances makes this approach ideal for high-aspect-ratio bioreactors, such as deep wells in multi-well plates.

If the oscillating mixer is not solid but instead a pressurized, thin-walled, gas-permeable bulb, the gas leaving the bulb is immediately mixed into the fluid flowing past it. In this configuration, gas transfer rates can exceed those of simple hollow fiber oxygenators because transfer occurs through both radial diffusion from the bulb surface into the annulus and convective transport of deoxygenated medium into the region immediately adjacent to the bulb surface and oxygenated media back into the region above or below the bulb. Gas flux across the bulb wall may include both convective and diffusive components, with the convective term controllable by adjusting the internal pressure of the bulb and the bulb velocity, and diffusion rate set by the bulb wall thickness and material properties. Furthermore, if the bulb is elastomeric and does not have internal radial constraints (e.g. an internal sponge structure or radial spokes), the internal gas pressure also controls the bulb's diameter, allowing mixing rates to be tuned dynamically as the medium viscosity increases with rising cell density. Gases generated within the medium, such as carbon dioxide, can likewise diffuse into the bulb, enabling the device to function as a bidirectional gas exchanger. The controlled flow of a gas mixture through the bulb enables active regulation of the partial pressures of carbon dioxide, oxygen, and possibly nitrogen or an inert gas inside the bulb, thereby permitting fine control of CO2 levels and thus the pH of the culture. In addition to the bulb, control of the gas partial pressures and flow rates in the headspace can also be used for additional gas exchange. The gas in both the bioreactor headspace and within the bulb can be humidified to eliminate evaporation of water from the culture media. Gas exiting the headspace and the bulb can be analyzed to quantify the metabolic activity of the cells within the bioreactor, including the generation of volatile compounds such as ethanol, methanol, hydrogen sulfide, ammonia, and a variety of nitrogen-and sulfur-bearing compounds. The mixer bulb and the mechanism that oscillates it can be designed so as to not interfere with either the delivery or removal of fluid from the bioreactor or the sensors within it.

FIG. 1 provides an overview of the invention that contains these features to create a displacement membrane gas exchanger and mixer (DM-GEM) that can be inserted into a well of a deep well plate to function as a bioreactor or chemostat. The key inventive feature is the simultaneous use of an oscillating, gas-permeable displacement object (a “bulb”) within a high-aspect-ratio cylindrical bioreactor (like a deep well plate well) to achieve two critical functions: efficient fluid mixing and gas exchange. The “mixer” is a smooth object, such as a sphere, whose cross-sectional area is slightly smaller than the bioreactor vessel. Its vertical oscillation, determined by the actuator and controller, displaces liquid, forcing it through the narrow annulus between the mixer and the wall. This motion creates a combination of laminar flow in the annulus (minimizing shear) and vortices in the bulk media (promoting rapid, large-scale mixing). This is a novel, mechanically simple approach ideal for high-aspect-ratio vessels where traditional stir bars are ineffective due to shear or poor vertical mixing. Because the displacer is fabricated as a pressurized, thin-walled, gas-permeable bulb (e.g., from PDMS or a nano-or microporous material), the device integrates gas exchange, with controlled delivery of mixed gassed into the bulb and analysis of gases leaving the bulb. As the bulb moves, the gas it transfers to the medium is immediately mixed by the fluid flow past its surface, minimizing local supersaturation and nucleation of bubbles that when detached trigger foaming. The cyclic movement ensures that the gas-exchange surface is constantly in contact with deoxygenated media, optimizing gas transfer. This is a key contrast to passive hollow fiber systems, which can be limited by diffusion and prone to bubble nucleation. The use of an elastomeric bulb allows the internal gas pressure to control its diameter, which dynamically adjusts the annular gap width. This, in turn, tunes the mixing rate to compensate for changes in medium viscosity as cell density increases.

According to the invention, the DM-GEM is specifically designed to overcome the challenges of mixing and gas exchange in deep wells of multi-well plates, which suffer from a small surface-to-volume ratio (limiting headspace gas exchange) and size constraints that limit conventional stirring/sparging. In addition, the large, slow-moving displacement mixer creates more uniform fluid velocities compared to small, high-speed impellers, minimizing the damaging shear forces on suspended cells. Further, by using a membrane bulb, the invention avoids the excessive foaming caused by direct gas sparging in protein-rich media. The mixing process ensures rapid integration of transferred gas, optimizing mass transfer. The gas-permeable bulb can function as a bidirectional gas exchanger, allowing oxygen delivery into the medium and removal of metabolic gases like carbon dioxide, and volatile organic compounds (VOCs). The controlled flow of gas through the bulb permits fine regulation of partial pressures, which can be used to control the dissolved gas levels and culture pH of the culture medium. The device can be implemented in a cartridge format, making it fully compatible with commercial well-plate systems and easily manufactured or serviced in large quantities.

Specifically, the bioreactor system in one aspect comprises a bioreactor vessel configured to contain a culture medium; and a displacement membrane gas exchanger and mixer (DM-GEM) configured to be positioned within the vessel, wherein the DM-GEM comprises a displacement member having an outer cross-sectional area smaller than an inner cross-sectional area of the vessel, thereby defining a fluid annulus between the displacement member and a wall of the vessel, wherein the displacement member has a gas-permeable surface defining an interior chamber therewith; and an actuator mechanism coupled to the displacement member and configured to vertically oscillate the displacement member through a substantial portion of the culture medium in the vessel. The vertical oscillation of the displacement member displaces the culture medium through the fluid annulus between the displacement member and the vessel wall to mix the medium while simultaneously exchanging gas across the gas-permeable wall of the displacement member.

In various embodiments, the gas-permeable surface of the displacement member is configured to permit diffusion of oxygen from the interior chamber into the culture medium and diffusion of carbon dioxide and VOCs from the culture medium into the interior chamber.

In various embodiments, the displacement member is an extensible bulb formed of an elastomer, and a diameter of the elastomeric bulb varies in response to gas pressure within the interior chamber.

In various embodiments, the vertical oscillation of the displacement member is configured to force the culture medium to flow through the annulus between the displacement member and the vessel wall; generate laminar flow within the annulus to minimize shear forces on suspended cells; and generate vortices and turbulent mixing in the wake region above and below the displacement member to promote large-scale mixing throughout the culture medium.

In various embodiments, the actuator mechanism is an electromagnetic actuator comprising a solenoid and at least one permanent magnet coupled to the displacement member, wherein current in the solenoid is controlled to generate the vertical oscillation of the displacement member.

In various embodiments, gas pressure within the interior chamber is controlled to regulate at least one of a gas transfer rate, a mixing intensity, and a shear rate experienced by cells.

In various embodiments, the displacement member is oscillated with a waveform selected to control a shear rate and mixing within the culture medium.

In various embodiments, gas transfer is bidirectional, permitting delivery of oxygen into the medium and removal of carbon dioxide from the medium. Gas composition, pressure, flow rate, and humidity within the displacement member may be actively controlled to regulate dissolved gas concentrations and pH. Mixing intensity and shear exposure may be tuned by adjusting displacement amplitude, frequency, velocity, or effective cross-sectional area of the displacement body.

In various embodiments, the system further comprises a gas management unit configured to deliver a gas mixture into the culture medium via diffusion and/or convection across the gas-permeable wall of the displacement member; receive metabolic gases from the culture medium via diffusion into the interior chamber of the displacement member; and/or dynamically alter dimensions of the displacement member to adjust a width of the fluid annulus and tune a mixing rate.

In various embodiments, the system further comprises a gas supply fluidly coupled to the interior chamber of the displacement member to deliver the gas mixture with a controlled gas composition; a headspace coupled to the vessel and configured to control gas partial pressures and flow rates in a headspace of the bioreactor vessel for additional gas exchange; and/or a humidification unit to humidify gas delivered to both the displacement member and the headspace to prevent evaporation of the culture medium.

In various embodiments, the DM-GEM further functions for temperature control, by regulating the temperature and flow rate of the gas delivered to the interior chamber of the displacement member to control the temperature of the culture medium.

In various embodiments, the DM-GEM is implemented as a cartridge configured for insertion into a well of a multi-well plate, wherein the cartridge further comprises coaxial conduits including a fixed inner tube delivering gas to the interior chamber and a movable outer tube removing gas from the interior chamber and controlling the vertical position of the displacement member.

In various embodiments, the bioreactor vessel is one well of a multi-well plate, and the system further comprises a plurality of displacement members operating in parallel in respective wells.

In various embodiments, the system further comprises one or more sensors configured to measure at least one of pH, temperature, dissolved oxygen, carbon dioxide, lactate, optical density, and metabolite concentration in the culture medium.

In various embodiments, the system is further configured for perfusion operation, including a means for continuous or intermittent removal of spent media and replacement with fresh media while retaining suspended cells in the bioreactor vessel.

In another aspect, the bioreactor system comprises a bioreactor vessel configured to contain a culture medium; a gas-permeable member dimensioned to be moved vertically within the vessel to define a fluid annulus between the gas-permeable member and a wall of the vessel; and a drive mechanism operably coupled to the gas-permeable member, and configured to cycle the vertical position of the gas-permeable member to displace the culture medium and promote mixing while simultaneously facilitating gas diffusion and/or convection across a surface of the gas-permeable member.

In various embodiments, the gas-permeable member is an elastomeric bulb.

In various embodiments, the system further comprises a means for adjusting the internal pressure of the gas-permeable member to modify dimensions of the gas-permeable member, thereby controlling a width of the annulus and dynamically tuning a rate of displacement mixing.

In various embodiments, the vertical movement of the gas-permeable member is configured to generate laminar fluid flow through the annulus to minimize shear forces on cultured cells in the culture medium; and/or optimize turbulent fluid flow (vortices) in bulk culture medium upstream and downstream of the gas-permeable member to promote homogenization.

In various embodiments, the system further comprises a gas management system configured to flow a gas mixture through an interior of the gas-permeable member to enable bidirectional gas exchange, including delivery of oxygen and removal of carbon dioxide.

In various embodiments, the drive mechanism comprises a mechanical crank, a pneumatic piston, a pneumatic bellows, and/or an electromagnetic actuator.

In yet another aspect, the bioreactor system comprises a vessel configured to contain a culture medium; a movable displacement body positioned at least partially within the vessel; an actuator configured to move the displacement body reciprocally along a substantially vertical path within the vessel so as to displace at least a substantial portion of the culture medium and thereby mix the culture medium; and a gas exchange interface integral to or associated with the displacement body and configured to facilitate exchange of at least one gas between the gas exchange interface and the culture medium in the vessel during the movement of the displacement body, wherein mixing of the culture medium and gas exchange occur concurrently as a result of movement of the displacement body within the vessel.

In various embodiments, the displacement body includes or is associated with the gas exchange interface, such as a gas-permeable wall or membrane, that permits transfer of gases between a gas source and the liquid culture medium during movement of the displacement body. As the displacement body traverses the vessel, substantially all portions of the liquid culture medium are periodically brought into proximity with the gas exchange interface, enhancing gas transfer efficiency and homogeneity.

In various embodiments, the movable displacement body has a cross-sectional shape comprising a spherical, spheroidal, cylindrical, or polygonal shape.

In various embodiments, the gas exchange interface comprises a gas-permeable portion of the displacement body.

In various embodiments, the gas exchange interface is configured to permit transfer of oxygen into the culture medium and transfer of carbon dioxide out of the culture medium.

In various embodiments, the movement of the displacement body causes liquid flow through a confined flow region between the displacement body and a wall of the vessel.

In various embodiments, the confined flow region produces a first flow regime adjacent to the displacement body and a second flow regime remote from the displacement body, the first and second flow regimes being different.

In various embodiments, the movement of the displacement body mixes the culture medium while limiting shear stress experienced by cells suspended in the medium.

In various embodiments, the location of the displacement object over time can be specified by a particular periodic waveform, such as sinusoidal, ramps with different up and down rates, or waveforms with intervals with no vertical motion.

In various embodiments, at least one of displacement amplitude, displacement frequency, displacement velocity, or effective displacement cross-section of the displacement body is adjustable during operation.

In various embodiments, gas transfer across the gas exchange interface is regulated by controlling gas pressure, gas composition, or gas flow rate.

In various embodiments, the displacement body is deformable such that an effective cross-sectional dimension of the displacement body varies during operation.

In various embodiments, the actuator comprises at least one of an electromagnetic actuator, a pneumatic actuator, a hydraulic actuator, a mechanical linkage, and a motor-driven actuator.

In various embodiments, the displacement body and actuator are integrated into a removable cartridge configured to be inserted into the vessel.

In various embodiments, the system further comprises a plurality of vessels each having a respective displacement body, enabling parallel operation of multiple bioreactors.

In various embodiments, the system further comprises control of at least one of dissolved oxygen, carbon dioxide and lactate concentration, pH, and temperature of the liquid culture medium, and/or the humidity in the headspace or the displacement member.

In various embodiments, the system further comprises one or more sensors configured to monitor at least one parameter of the liquid culture medium or gas exchanged through the gas exchange interface.

The systems may be implemented as compact cartridges insertable into individual wells of a multi-well plate, enabling massively parallel operation. The invention is compatible with batch, fed-batch, and continuous or intermittent perfusion modes, and may incorporate sensors and control systems for automated operation.

In one aspect, the invention relates to a system for parallel bioreactor operation, comprising a well plate comprising a plurality of wells; a plurality of cartridge units, each cartridge unit corresponding to one of the plurality of wells and comprising a movable displacement body, an actuator configured to impart reciprocal motion to the displacement body within its corresponding well, and a gas exchange interface integral to or associated with the displacement body; and a controller operably coupled to the plurality of cartridge units and configured to independently or synchronously control movement of each displacement body and gas delivery to each gas exchange interface.

In various embodiments, each cartridge unit further comprises at least one integrated sensor for monitoring a condition within its corresponding well.

In various embodiments, the actuators for the plurality of cartridge units are configured to move in unison, oscillating a corresponding array of displacement bodies relative to the well plate.

In various embodiments, the system further comprises a gas management unit configured to supply a controllable gas mixture to the gas exchange interface of each displacement body.

In various embodiments, the gas management unit is further configured to humidify the controllable gas mixture.

In various embodiments, the system further comprises a cell separation means that enables continuous or intermittent perfusion culture with partial removal of condition media and its replacement with fresh media while minimizing cell loss.

In another aspect, the invention relates to a method for culturing cells within a bioreactor vessel containing a culture medium, comprising a gas-permeable displacement member within the culture medium, wherein the displacement member has a gas-permeable surface defining an interior chamber therewith; oscillating the displacement member vertically relative to a wall of the vessel to impart movement to the culture medium; and exchanging gases between the interior chamber of the displacement member and the culture medium across the gas-permeable surface of the displacement member during the oscillation.

In various embodiments, the bioreactor vessel is a deep well of a multi-well plate.

In various embodiments, the oscillating and exchanging steps are performed to provide adequate vertical mixing and gas transfer despite the small surface-to-volume ratio of the well.

These and other aspects of the invention are further described below. Without intent to limit the scope of the invention, examples and their related results according to the embodiments of the invention are given below. Note that titles or subtitles may be used in the examples for convenience of a reader, which in no way should limit the scope of the invention. Moreover, certain theories are proposed and disclosed herein; however, in no way, whether they are right or wrong, should they limit the scope of the invention so long as the invention is practiced according to the invention without regard for any particular theory or scheme of action.

### Displacement Mixing

FIG. 1 shows one embodiment of the invention, wherein a displacement mixing and gas exchange bulb is oscillated vertically within a deep well or other bioreactor vessel to provide two separate functions: 1) mixing of the suspended cells, nutrients, waste products, other chemicals, and gases in the fluid contained by the well, and 2) the exchange of gases between the interior of the gas-permeable bulb and the fluid in which the cells are suspended. In this embodiment, media and possibly cells are delivered to the well through a media/cell delivery tube, and the level of media within the well can be controlled by intermittent or continuous removal of fluid and possibly cells by means of a level-setting withdrawal tube, and an external media and cell analyzer that contains a pump. Under control of the gas management system, a gas mixture is delivered to the interior of a gas-permeable bulb, either by diffusion through the material of the bulb wall or by passage through microscopic pores in the bulb wall. A mechanical mechanism governed by an actuator/controller system is used to drive the vertical oscillations of the bulb. A tube delivers a gas mixture to the headspace, and another tube removes from the headspace gas that now contains a different mixture of gases, most notably with less oxygen and more carbon dioxide and VOCs. The gas removed from the headspace and/or the bulb can be analyzed, for example to measure levels of carbon dioxide or volatile organics such as ethanol. Depending upon the humidity and temperature of the gas in the input stream, the gas leaving the headspace may have a higher or lower humidity. A deep sample removal tube, in this embodiment oscillating with the bulb, allows continuous or intermittent removal of samples of media and cells for analysis. Sensors within the bioreactor measure pH, temperature, O₂, glucose, lactate, or other analytes. The mixer bulb and the actuator mechanism that oscillates it can be designed so as not to interfere with either the delivery or removal of fluid from the bioreactor or the sensors within it.

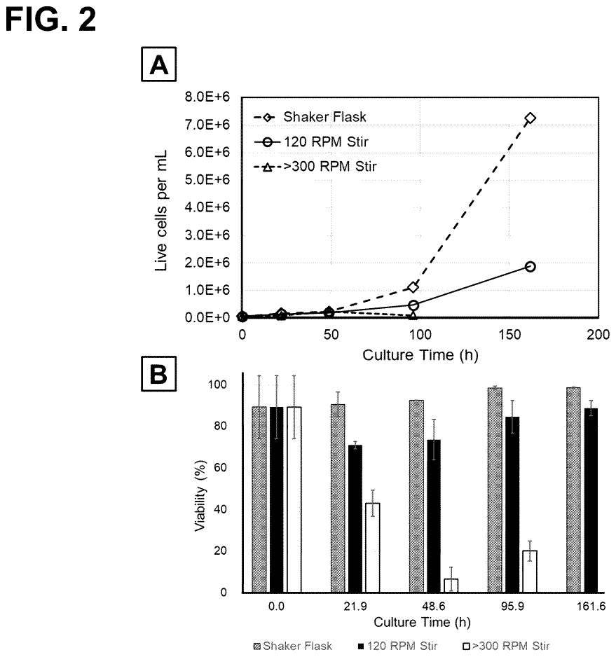

This invention provides an efficient means to mix the media and exchange gases in a bioreactor with a high aspect ratio (diameter to height) that is typical in the narrow yet deep wells in deep 48-well plates. This is in contrast to the limited mixing that can be achieved with stirring from the bottom with a stir bar or impeller and restrictions in the rate of gas exchange at the headspace-fluid interface that result from the small surface-to-volume ratio of the high-aspect-ratio wells. FIG. 2 presents the growth curve (panel A of FIG. 2) and viability (panel B of FIG. 2) for Chinese hamster ovary (CHO) cells grown in a shaker flask compared with average values from three 1.5 mL wells. The cells were cultured at 37° C., 8% CO₂. The stir bar was 7×2 mm, operating at speeds of 120 RPM and >300 RPM. The cell concentration and viability were measured using a Countess optical cell counter (equal 10 μL samples from each well were added to 30 μL of trypan blue for analysis), with two measurements per data point. These data demonstrate that CHO cells grow faster in a shaken flask than in a deep well mixed by a magnetically driven stir bar. Much higher stir speeds would have been required to create an adequately deep vortex to get good gas transfer to the bottom of the well, and that in turn would have complicated control of the fluid level and increased the shear forces that the cells experienced and reduced their viability. The use of a sparger to deliver gas through a large number of rising bubbles would generate foam that would compromise the metabolic activity of cells trapped in the foam, complicate level-setting fluid withdrawal, or require the use of anti-foam agents, which can adversely affect cellular metabolism and even damage the cells.

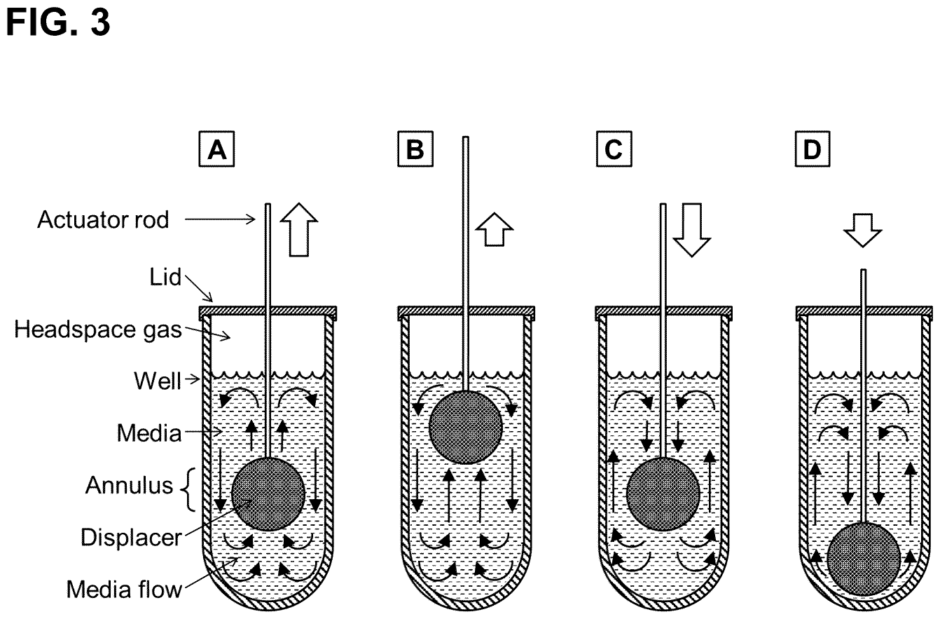

FIG. 3 demonstrates how the vertical oscillation of a spherical displacer mixes the media within the well by forcing media through an annulus and creating vortices that mix the majority of the culture media in a single stroke. With this approach, the “mixer” is a smooth object, such as a sphere with a cross-sectional area somewhat smaller than that of the bioreactor vessel, that displaces liquid upward through the annulus as the mixer descends and downward as the mixer rises. The vertical position of the bulb could oscillate with a range of amplitudes, with the maximum range being the top and bottom of the fluid-filled chamber.

The ratio of the cross-sectional area of the fluid-filled annulus between the mixer and the bioreactor wall to the cross-sectional area of the bioreactor itself, together with the oscillation frequency, the stroke length, and the viscosity of the medium, determine not only the rate and effectiveness of the mixing rate, but also the mechanical force and hence power required to drive mixer displacement, with higher frequencies and amplitudes requiring more power to overcome the viscous resistance of the media and cells. FIG. 3 describes qualitatively the changing fluid velocities in a displacement mixer as the displacer is periodically raised and lowered.

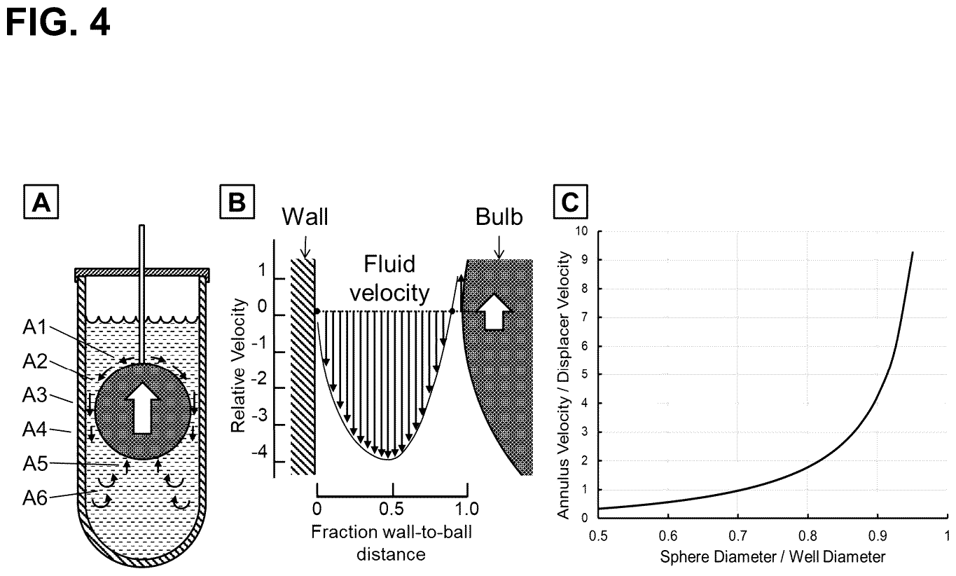

Panel A of FIG. 4 shows the geometry of the mixer created by a solid sphere. As this ball moves upward, the media below it is displaced and forced downward through the annular gap between the stationary cylinder wall and the moving ball. As shown by the small arrows, the initial flow at the leading edge of a rising spherical displacer ball (A1) must have a radial component since it would likely have the same resistance to flow in all directions and the space above is fully confined by the cylindrical vessel boundary, causing the fluid to split apart rdially at the center of displacement. As the fluid rounds the front shape of the displacer ball (A2), its direction will become more and more opposite to that of the motion of the displacer ball motion and will exhibit acceleration as it enters the narrowing annulus. At the narrowest point in the annular gap (A3), the flow will have its highest velocity exactly opposite to the motion of the displacer ball, with its relative velocity being a function of the annular gap area. As the fluid is ejected through the annular gap (A4), the motion will switch rapidly to inertially dominated flow with rapid deceleration at the interface of the opposite flow stream and the entrained flow stream on the back side of the displacer ball (A5) flowing in the same direction. This will cause vortex shedding and rapid mixing (A6) on the back side of the displacer ball.

Panel B of FIG. B plots the fluid velocity in the annulus for an upward moving displacer ball. A simplified velocity profile defined by the vertical arrows in panel B of FIG. 4 would be a curve that includes flow around the displacer ball going in the opposite, downward direction through the bulk of the annular space, shown as the peak relative velocity of −4 midway between the cylinder wall and the ball. Note that because of the no-slip boundary condition associated with laminar flow of an incompressible Newtonian fluid, the velocity of the fluid at the surface of the displacer matches that of the upward-moving displacer, mildly distorting the parabolic flow profile associated with static Poiseuille flow. The fluid touching the cylinder wall on the left has zero velocity, shown by the leftmost dot. The fluid touching the surface of the displacement ball on the right must have the same velocity as the upward-moving displacement ball, i.e., a velocity of +1 shown by the single upward-pointing arrow at the right. There is a no-flow position as shown by the dot to the immediate left of the upward pointing arrow.

Hence, the combination of annular flow (A3 in FIG. 4A) and deep vortex shedding (A6), the periodic sweeping of these zones over the depth of the well, and their relative location exchange upon direction reversal all contribute to the efficiency of displacement mixing and gas exchange.

Panel C of FIG. 4 illustrates the dependence of the average fluid velocity in the annulus between the sphere and the cylinder wall as a function of ball size. (A reasonable approximation for the parabolic (Poiseuille) flow in the annulus would have the average flow as two-thirds of the peak flow.) For a ball that is one-half the diameter ot the cylinder (leftmost end of the curve), the cross-sectional area of the ball is one-fourth that of the cylinder, so that the annular area is three times that of the sphere. Hence, the fluid velocity in the annulus is one-third that of the ball. For a sphere whose area is one-half that of the cylinder, the annulus and the sphere have the same areas and hence the annular peak velocity is equal and opposite to that of the sphere. For larger spheres, the annular velocity increases. The ratio of the cross-sectional area of the ball relative to that of the enclosing cylinder determines the height of the positive peak in panel A of FIG. 4: If the ball area is greater than half of the cylinder cross-sectional area, the fluid in the annular gap will have a higher velocity than that of the ball. We find that the ideal diameter ratio should be somewhere between 0.8 and 0.9, i.e., area ratios of 0.6 to 0.8, such that the annular fluid velocity is about two to four times the velocity of the displacer ball.

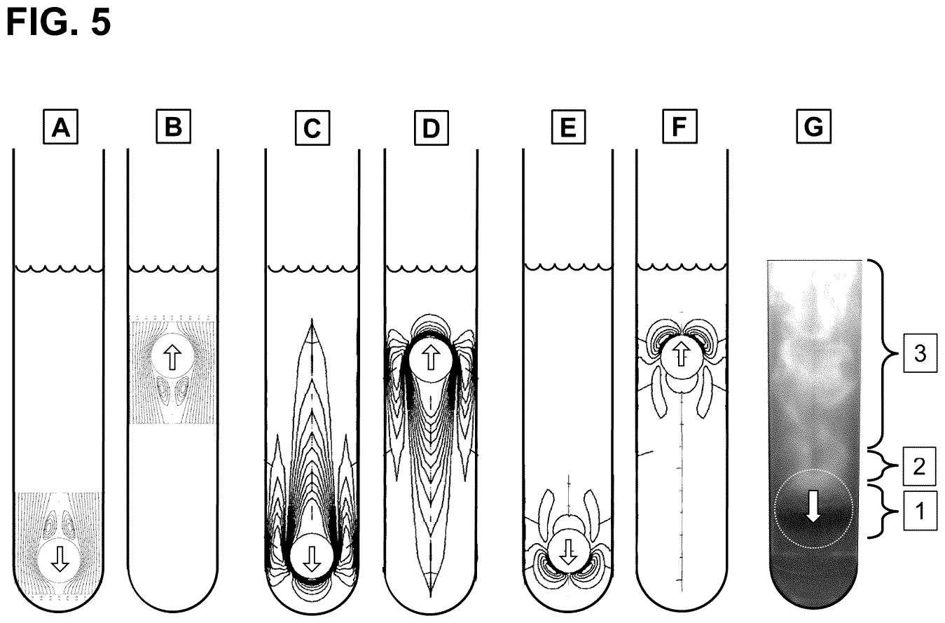

The actual flow profile is more complicated than shown in panel B of FIG. 4, since the flows have both radial and axial components. FIG. 5 illustrates the fluid flow from a sphere moving in a fluid-filled tube. The requirement for both a static boundary condition of zero fluid velocity at the bioreactor wall and a moving boundary condition with the fluid and sphere velocities being equal at the surface of the moving sphere complicates the computation of the flow field over that of a ball fixed in a stationary tube filled with moving fluid. Adapted from Zheng et al. [1], panels A-B of FIG. 5 show streamlines of the Newtonian flow for downward and upward motion of the bulb. Panels C-D of FIG. 5 report the axial velocity contours, demonstrating the depth into the tube that the fluid moves with a significant velocity. Panels E-F of FIG. 5 provide the radial velocity contours and indicate how the mixing is three-dimensional. Note that the images used in panels A-F of FIG. 5 were for steady flow of a sphere in a long cylindrical tube and hence did not reflect the changes with flow patterns that would occur as the downward moving ball approaches the bottom of the well, or as the upward moving ball approaches the surface of the liquid in the bioreactor.

Consistent with these flow distributions, panel G of FIG. 5 shows a photograph of the disturbance of mica particles suspended in stationary water as a sphere is being pushed downward in a single stroke. The cyclic creation and dissipation of the vortices both above and below the displacer will dramatically increase the mixing throughout the entire media as compared to what could be achieved with one or more propellers or stir bars operating at different depths of a bioreactor or well.

Given the size of the resulting vortices and the relatively slow movement of the large mixer, the fluid velocities in the annulus will be more uniform than those produced by small, high-speed impellers or stir bars, thereby minimizing the shear forces encountered by the suspended cells and maximizing the generation of large-scale wake vorticies. It is central to this invention that the high-velocity flow in the annulus is laminar (Region 1 in panel G of FIG. 5), which is important for not applying excessive shear forces to the cells, but it also minimizes mixing across different radii within the annulus, whereas above or below the bulb the flows are turbulent and generate random vortices that promote large-scale mixing (Region 3 in panel G of FIG. 5). There is a transition zone between the laminar and turbulent flows (Region 2 in panel G of FIG. 5). The vertical movement of the displacer ensures that these three zones move over time. The cycle time of this motion can be chosen to optimize the mixing depending upon factors such as cell density, the effective viscosity of the media, and the maximum allowable chemical and shear gradients. The mechanical simplicity of displacing a mixer over large vertical distances makes this approach ideal for high-aspect-ratio bioreactors, such as deep wells in multi-well plates. The slow vertical motion of a large displacer with a narrow, moving annular region accomplishes what would be difficult with a small, fixed, high-speed impeller or stir bar.

The creation of vortices by a ball moving in a large tank depends upon the speed of the ball. In this case, the Reynolds number is proportional to the ratio of the speed at which the ball is moving through the fluid divided by the diameter of the ball, which is the characteristic dimension of the system being studied, i.e., a ball in a large tank. Taneda (1956) [2] showed that there were no significant vortices for Reynolds numbers below 30, and prominent vortices for Reynolds numbers from 75 to 133. In our invention, the three characteristic dimensions are the diameter of the cylindrical bioreactor, the diameter of the ball, and the width of the annular gap between the ball and the wall of the cylinder. The relevant velocity is the local fluid velocity, and hence there will be significant variation in the Reynolds number between the fluid above, adjacent to, and below the moving ball and near the bioreactor bottom, walls, and air-liquid interface at the top of the fluid. The media immediately adjacent to the ball is stationary with respect to the moving ball and has a zero Reynolds number, even though the ball and the immediately adjacent media are both moving vertically. The media immediately adjacent to the stationary wall of the bioreactor is also stationary. As a result, in the frame of reference of the bioreactor, the bulk media near to the wall is moving in the opposite direction to the bulk media near the ball, as shown in panel B of FIG. 4. Near the displacer, viscous forces dominate, leading to lower Reynolds numbers, while far from the ball inertial forces dominate, leading to higher Reynolds numbers. The higher the Reynolds number, the more likely is the formation of vortices, eddies, and either chaotic or turbulent mixing. In the annular gap, the Reynolds number is low and the flow is laminar.

With this approach, the generation of the vortices will be determined by the velocity of the displacer, the gap between the displacer and the bioreactor wall, and the viscosity of the fluid being mixed. Because there will be a steep gradient in fluid velocity from one side of the gap to the other, there will also be a significant shear rate in the fluid in the gap. As we discussed above with regard to the efficiency of mixing and any shear-related cell damage, it would be ideal were it possible to control independently the fluid velocity and the shear rate. Given the steep dependence of the flow velocity in the annular region on the width of the gap, we recognize that small changes in the diameter of the bulb could lead to significant changes in both shear rate and vortex formation. As we will discuss below, displacement mixing can be tuned to specific viscosity ranges by changing either the motion profile or the mechanical profile of the displacement object.

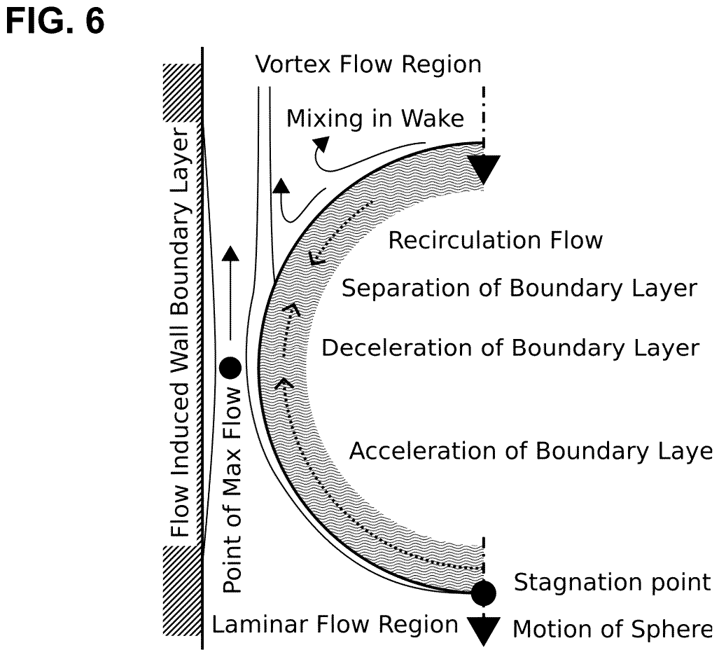

Providing more precise detail than panel G of FIG. 5, FIG. 6 shows a diagram of the flow boundaries generated by the movement of a sphere in a vertical tube. The combination of laminar flow beneath the leading face of the downward movement and the recirculating flow above the trailing face are of particular importance to the desired function of the system. It is critical to the function of this invention that the flow regime zones exchange position as the motion reverses.

On the leading face of the mixing object, the flow is largely laminar and no significant mixing occurs. This laminar flow is important to get diffusive access to the entire reactor volume for gas transfer. Once the fluid passes the point of maximum velocity and the boundary layer separates from the mixing object, the recirculation flow in the wake of motion provides thorough mixing of that volume. When the mixing object reaches the end of its stroke length and changes direction, these flow regime zones flip, which ensures that gas exchange and mixing functions are presented to the entire volume of the reactor.

On the leading face, a stagnation point develops where forces are balanced so that there is near zero fluid movement relative to the mixing object. Reversing the direction prevents this stagnation zone from persisting for longer than one-half of the cycle time.

The laminar flow region along with the narrow annulus ensures that the entire leading-face volume comes in close proximity of the gas exchange membrane. The periodic reversal of the direction of motion and the exchange of the zones on the opposite faces of the moving object ensure that the entire bioreactor volume is properly gassed with a few strokes of the mixing object.

Recirculation flow in the wake of the moving object is not a stable flow state and therefore thorough chaotic mixing takes place in the area behind the motion of the mixing object. In the course of the vertical displacement cycle, the recirculating wake flow occurs alternately above and below the displacement object.

At the singular stagnation point, the no-slip boundary condition requires that there is no radial flow immediately at the surface, and since the displacement force is normal to the surface of the object, neither is there any axial flow, i.e., the definition of a stagnation point. The flow fields only develop with distance from the stagnation point: as the fluid is physically displaced by the motion, a shear line develops tangent to the surface of the object. This flow increases as the distance from the stagnation point increases.

At the stagnation point the dimension related to Reynolds number is as large as the containing vessel. In the annular space around the curvature of the sphere, the distance decreases, which prevents the Reynolds number from going significantly higher.

The speed at which the sphere moves influences the Reynolds number but does not reach speeds that would cause the flow on the leading face to become turbulent.

### Displacement Gas Exchange

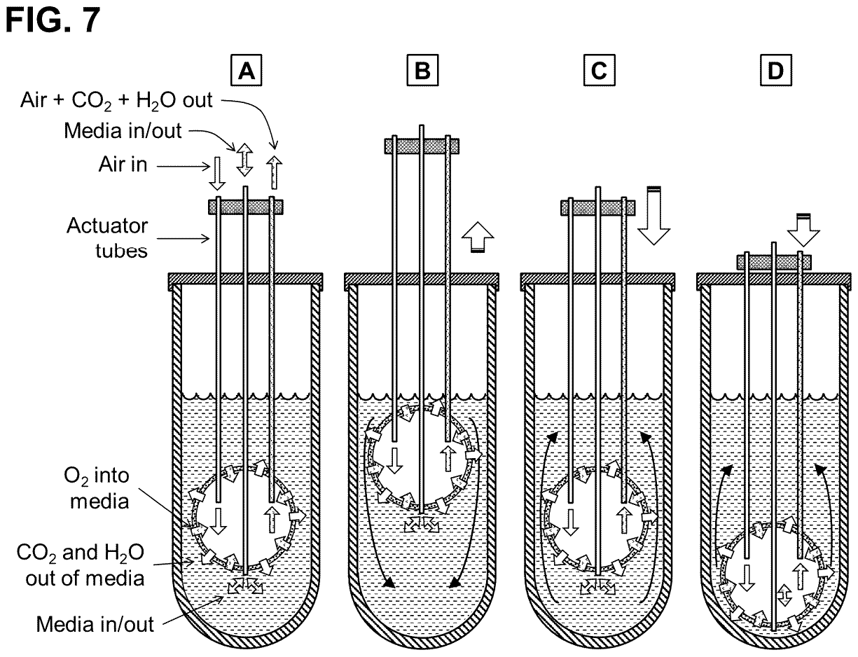

If the oscillating mixer is not solid but instead a pressurized, thin-walled, gas-permeable bulb, for example fabricated from PDMS or nano-or micro-porous material, the gas leaving the bulb is immediately mixed into the fluid flowing past it (FIG. 7). In this configuration, gas transfer occurs through both radial diffusion into the annulus and the fluid that is adjacent to the rest of the surface of the bulb, and convective transport of deoxygenated medium into the region immediately adjacent to the surface of the bulb and oxygenated media back into the region above or below the bulb. Gas flux across the bulb wall may include both convective and diffusive components, with the convective term controllable by adjusting the internal pressure of the bulb, the internal oxygen concentration, and the bulb velocity, and the diffusion rate determined by bulb wall thickness, the internal versus external oxygen concentration, and material gas-transfer properties. Gases generated within the medium, such as carbon dioxide, can likewise diffuse into the bulb, enabling the device to function as a bidirectional gas exchanger. The controlled flow of a gas mixture through the bulb enables active regulation of the partial pressures of carbon dioxide, oxygen, and possibly nitrogen or an inert gas inside the bulb, thereby permitting fine control of CO2 levels and thus the pH of the culture. In addition to the bulb, control of the gas partial pressures and flow rates in the headspace can also be used for additional gas exchange and pH control.

The delivery of dry gas via a gas-permeable bulb oscillating in a well can lead to water loss in the well, wherein the water either evaporates into the headspace gas and escapes through the interface between the sliding actuator tubes and the hole through which they pass or diffuses across the membrane into the interior of the bulb, where it is flushed out with the continuous or intermittent exchange of gas within the bulb. In order to control the humidity in both the headspaces and oxygenating bulbs of a multi-well bioreactor system, pumps or combinations of pumps and valves can deliver metered amounts of humidified gas mixtures to both the bioreactor headspace and the gas-exchange bulb. All of the bioreactors operating in a multi-well plate can operate at a fixed overpressure above atmospheric so that there is no possibility of gas crosstalk between bioreactors. Hence, it can be important that the gas in both the bioreactor headspace and within the bulb is properly humidified to eliminate evaporation of water from the culture media. Gas exiting the headspace and the bulb can be analyzed to quantify the metabolic activity of the cells within the bioreactor.

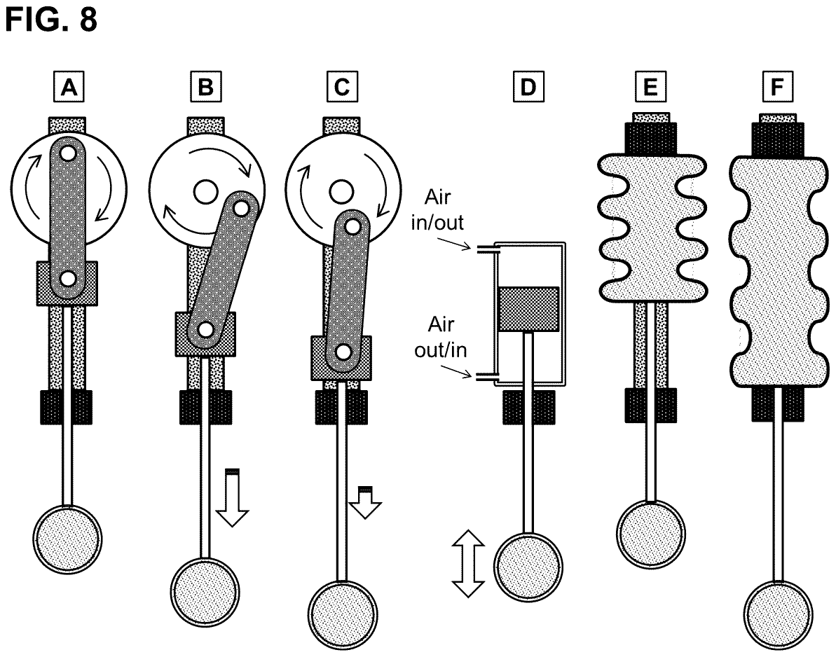

While semipermeable membranes have been used for gas exchange in miniature bioreactors, we know of no examples of the use of a moving, thin-walled, gas-permeable bulb serving as both a mixer and gas exchanger. To test this, we first used a mechanically rotating crank and connecting rod drive to oscillate the displacer vertically (panels A-C of FIG. 8). The vertical motion could also be driven by a pressurized piston (panel D of FIG. 8), a pneumatically or hydraulically controlled bellows (panels E-F of FIG. 8), or any number of other methods capable of providing periodic virtual motion.

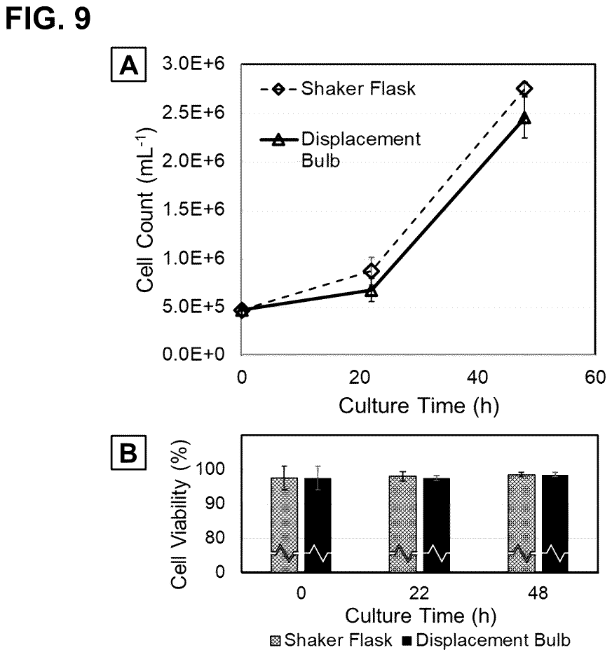

FIG. 9 demonstrates that a mechanically driven displacement mixer bulb can grow CHO cells to a density that is comparable to what can be achieved in a shaken flask. For these measurements, the bioreactor vessel is a 1 cm diameter glass test tube with a hemispherical bottom that contains 2.5 mL of media. A mechanical drive (panels A-C of FIG. 8) oscillated the bulb at 6 cycles/minute. Environmental air from outside of the incubator was delivered to the bulb at a pressure of 1 psi (˜6.9 kPA), with a flow rate of ˜20 mL/min. The apparatus was operated in a cell culture incubator at 37° C. with 8% CO₂. Note that the measured cell concentration in the tube does not account for likely evaporative loss from the media, through the bulb, and into the air being passed through the interior of the bulb.

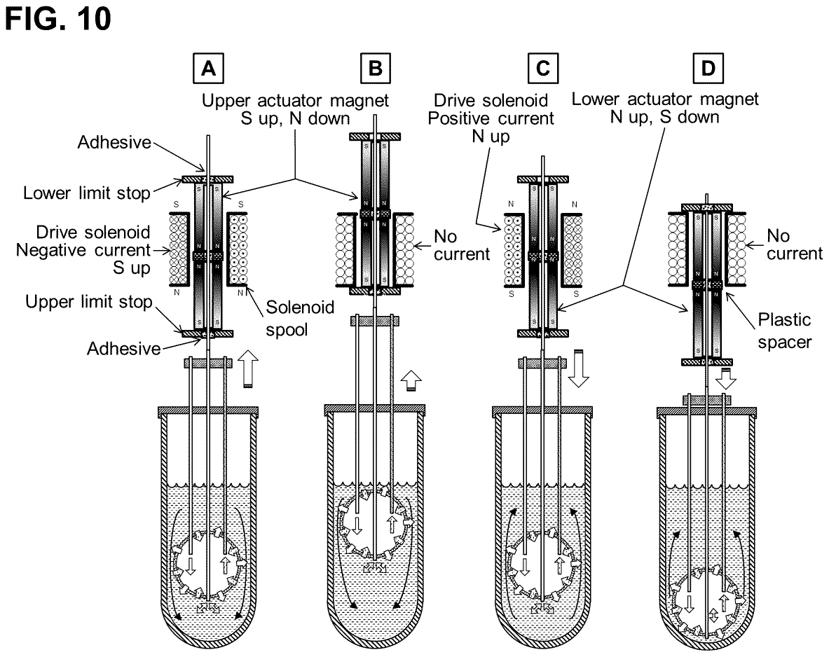

FIG. 10 shows a bistable electromagnetic actuator, an embodiment of this invention with a simple design that is easily controlled and reliable, and that uses a fixed solenoid coil and a pair of opposing rare earth permanent magnets to drive 12 mm vertical displacements of an 8 mm PDMS spherical displacer. The permanent magnets create a quadrupolar magnetic field that interacts with the dipolar magnetic field of the solenoid. As shown, the system operates in the near field of both the permanent magnets and the solenoid, where there is a smooth variation in force as a function of the vertical displacement of the permanent magnets relative to the solenoid. The forces can be understood qualitatively by considering the effective north and south magnetic poles at the ends of the solenoid interacting with the two south magnetic poles at the two ends of the permanent magnet pair and the two adjacent (and self-repelling) north magnetic poles at the center of the pair. As drawn, there are two stable states of the system, one for each sign of solenoid current. With solenoid current of one sign, the magnetic field from the solenoid acts primarily to lift the upper magnet out of the top of the solenoid (panel A of FIG. 10). The south magnetic pole of the solenoid, at the top, repels the south magnetic pole at the top of the permanent magnet pair and attracts the double north poles in the middle. Simultaneously, the north magnetic pole at the bottom of the solenoid repels the double north magnetic pole in the center of the permanent magnet pair and attracts the south magnetic pole at the bottom of the pair. Hence, all four of the pole-pole interactions produce an upward force.

When the current is reversed, these four forces reverse sign, driving the permanent magnet pair downwards. Hence, the total force applied to the pair of magnets is the sum of the forces acting on each magnet by the magnetic field from each of the windings of the solenoid. The length and diameter of the solenoid, the length and diameter of each magnet, and the magnet separation provided by the plastic spacer can all be adjusted to optimize the response of the magnets to the current in the solenoid by shaping force-versus-axial-displacement curves to adjust both the forces and the stability of these two stable states. While axial holes in each permanent magnet reduce the strength of the magnet as compared to a solid one, they provide significant design benefits with regard to mounting the magnets and coordinating gas transfer into and out of the bulb. Other magnetic configurations can be realized, and this one is presented as an example.

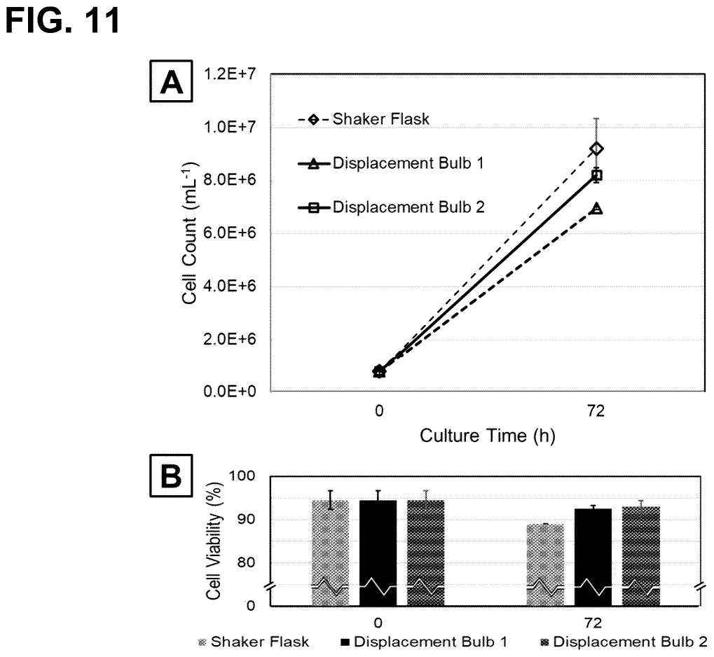

FIG. 11 shows the results of the first reduction to practice of the electromagnetic actuator. For these preliminary tests, the bottom half of the bulb was a thin PDMS membrane while the upper half was a solid PDMS hemisphere of the same diameter, operating in a glass test tube with an outer diameter of 12 mm. For these measurements, CHO cell subcultures were taken from a single culture of CHO cells grown in a flask on a shaker. The “Shaker Flask” subculture was continued for 72 hours in a flask on a shaker, and “Bulb” subcultures were grown in separate tubes over the same 72-hour period. Panel A of FIG. 11 shows the cell density (cells/mL) for these three cultures, while panel B of FIG. 11 shows cell viability of the subcultures, i.e., the percentage of live cells based on staining with trypan blue. For both graphs, the average of two measurements was used for each datapoint, and the error bars represent the standard deviation of the two values.

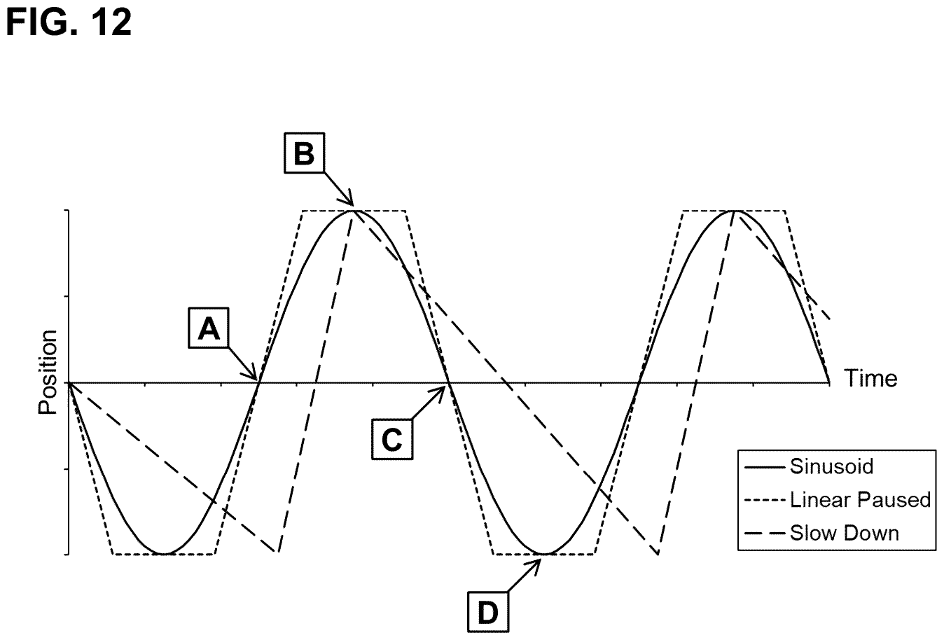

The electromagnetic actuator has the advantage of allowing a waveform-generating microcontroller to specify exactly the time dependence of the vertical position of the displacer. FIG. 12 shows how different drive waveforms could provide user-controlled upward versus downward velocities with pauses, or a smooth sinusoidal displacement or any of a number of different waveforms. Pulse-width modulation (PWM) can be used to control position and the speed with which the mixing ball moves inside the well. With one proven mixing protocol, the ball stays for two seconds in the lowest position and one second in the top position. Thirty percent of the maximum force is sufficient to hold the ball in the top position (no holding current is applied once the ball reaches the bottom-most position). With this protocol, there is insignificant heating of the solenoids.

For the most complete mixing, the stroke length of the displacer should be the fluid depth minus the reactor diameter. Since the displacement objects'dimensions are tied to the reactor dimensions, this will provide the same top and bottom margins as the side margins when using a spherical displacement object. For expandable objects, another parameter will need to be added to any calculation of the mixing rate.

With regard to oxygenation, the movement of the gas-exchange membrane through essentially the entire volume of the bioreactor effectively increases the ratio of surface area to volume for mass transfer of gas, since the displacement bulb makes close contact with the entire volume of the reactor when the displacement bulb is constructed from a gas-permeable material. The surface of the displacement bulb could be designed to be molecularly rough but hydraulically smooth to maximize gas transfer surface area but prevent boundary-layer stagnation of fluids.

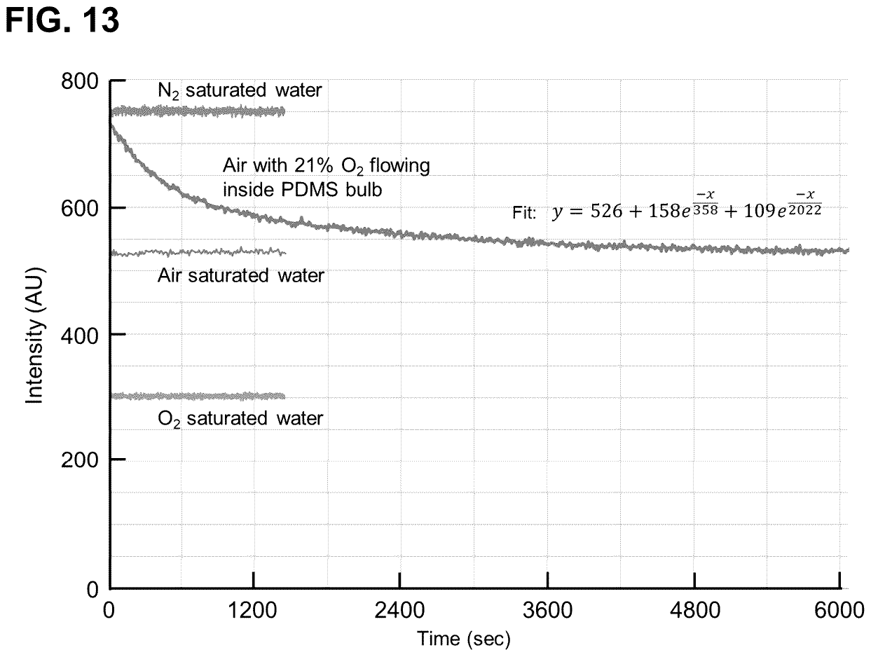

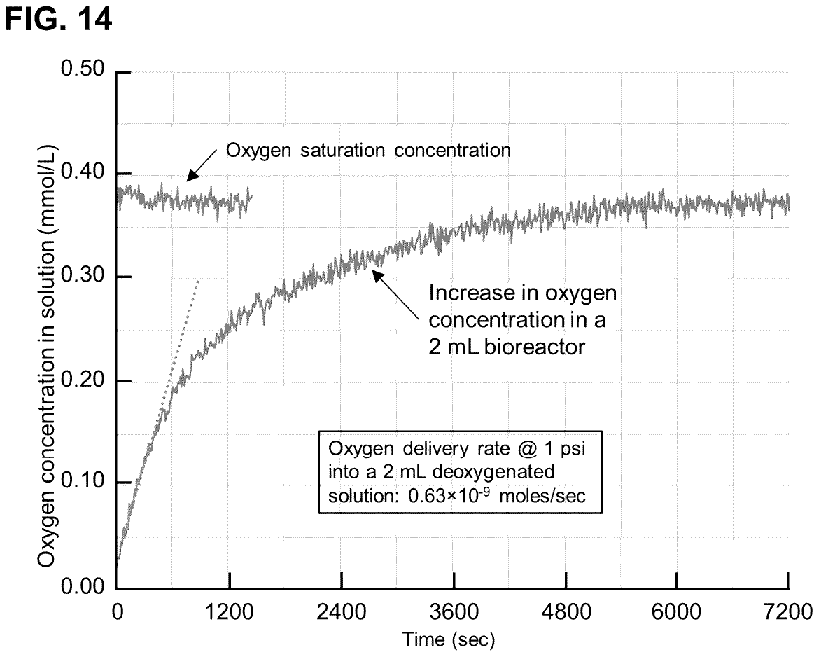

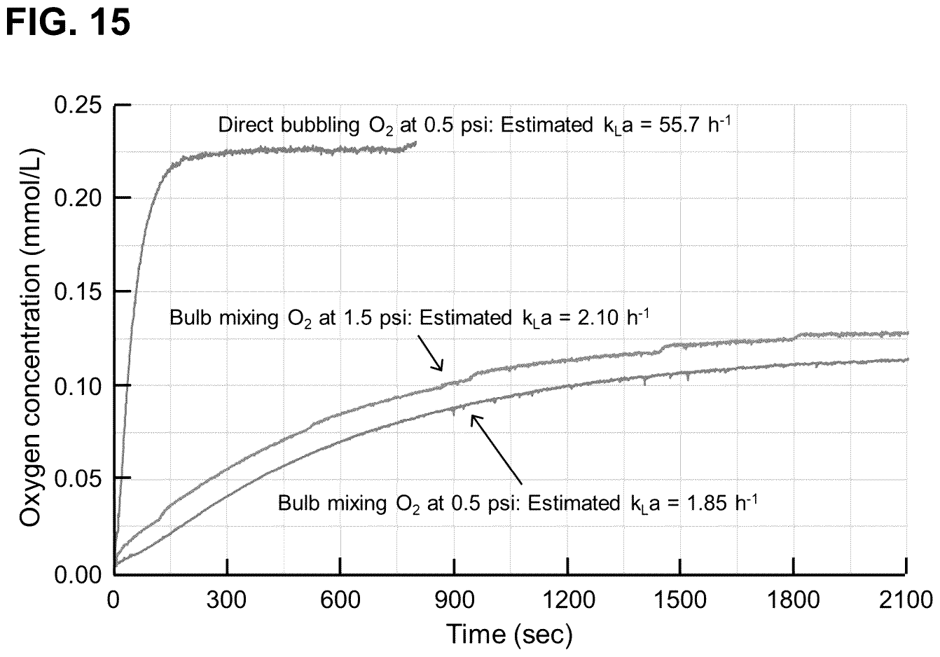

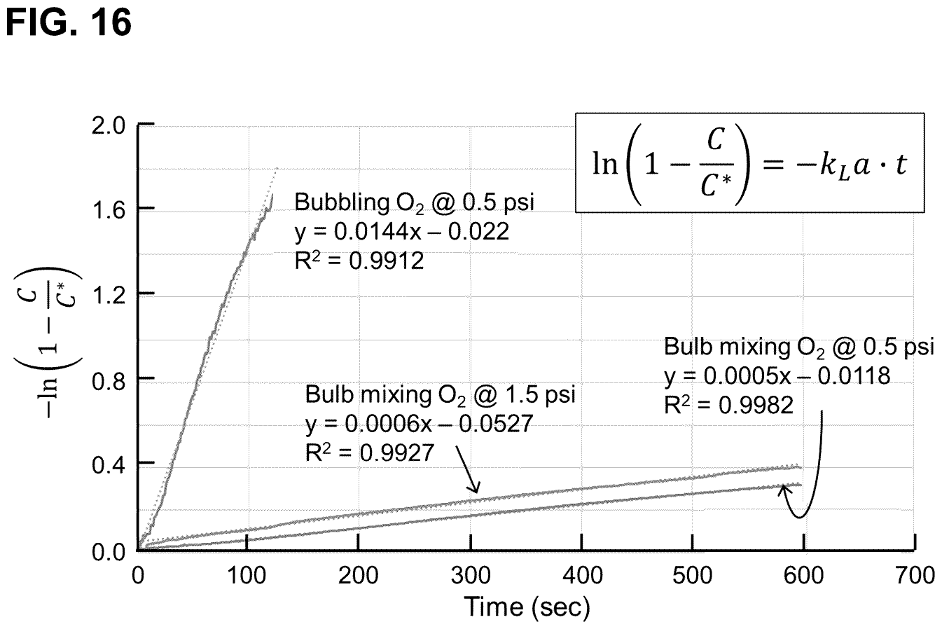

FIG. 13 plots the time dependence of the readout of a fluorescent oxygen sensor in the liquid above a moving PDMS displacement mixer bulb filled with 1 psi air, beginning with 22° C. water that was initially saturated with nitrogen. The intensity values for N₂, air, and O₂ saturated water define the possible range of intensities. The probe intensity can be converted into an O₂ concentration, such that FIG. 14 provides a quantitative measurement that can be used to determine the initial O₂ delivery rate at 1 psi. FIG. 15 allows comparison of measurements of oxygen delivered into a 2 mL bioreactor using a displacement membrane gas exchanger and mixer operating at 0.5 and 1.5 psi O₂ pressure, as compared to the direct bubbling of O₂ into the media. The k_{L}a values were determined as shown in FIG. 16 following Garcia-Ochoa [3]. The concentration of O₂ within the bulb and hence the diffusion rate through the PDMS are both determined by the absolute pressure within the bulb (14.5 versus 15.5 psi, i.e., a 6.9% difference), whereas the convective flow of O₂ through micropores in the PDMS membrane should be determined by the pressure difference across the membrane (0.5 vs 1.5 psi, i.e., a 200% difference). Given that the k_{L}a at 1.5 psi was 13.5% greater than that at 0.5 psi, we estimate that O₂ transport across the membrane is due to both diffusion and convection. Because the bulbs used in these measurements were fabricated by bonding a hemispherical PDMS shell to a matched PDMS hemisphere, we limited the pressure within the bulb to 1.5 psi lest we rupture the seal. Because we did not see nucleation of attached gas bubbles at the surface of the bulb at either pressure, we concluded that we could further increase the k_{L}a by increasing the pressure to increase both interior O₂ concentration and pressure-driven flow, using a thinner wall to decrease the flow resistance and increase the transport through the PDMS bulk, and increasing the fraction of the sphere's area that was membrane rather than solid PDMS. In the measurements in FIG. 15, we were not delivering O₂ to the headspace and hence did not achieve the same steady-state O₂ levels as we observed with direct bubbling, since O₂ delivered by the displacement membrane gas exchanger and mixer bulb was being lost into the unenriched headspace. While direct bubbling has a very high k_{L}a, we reiterate that direct bubbling is not a viable option in small bioreactors filled with media because of foaming, but in tests with water it provides an upper limit to the maximum O₂ delivery rate.

An alternative to a pressure-regulated gas control for one or more membrane gas exchanger bulbs would be to control the flow rate through each bulb. At 15 psi air (1.0E5 Pa), a flow rate of air into the bulb of at most 1.5 μL/min would be required to deliver 0.2 nmole/sec to the interior surface of the PDMS bulb, a rate that is fully consistent with the delivery that can be achieved with a miniature rotary planar peristaltic micropump (RPPM), which can readily pump between 1 and several hundred μL/min. (Should a higher density of cells be desired, the gas pressure in the bulb can be increased, air could be replaced with a higher concentration of O₂, the membrane thickness decreased, the bulb surface area increased, or the PDMS replaced with Teflon-AF.) Our 12-channel spiral RPPMs can readily meter out 15 psi gas in each of 12 channels. One advantage of a using a multi-channel pump to meter the delivery of gas to each well is that it avoids the problems encountered with passive gas splitters wherein a droplet of condensation could block one channel, which would prevent gas exchange with that bulb and increase the rate of gas delivery and exhaust for the other channels.

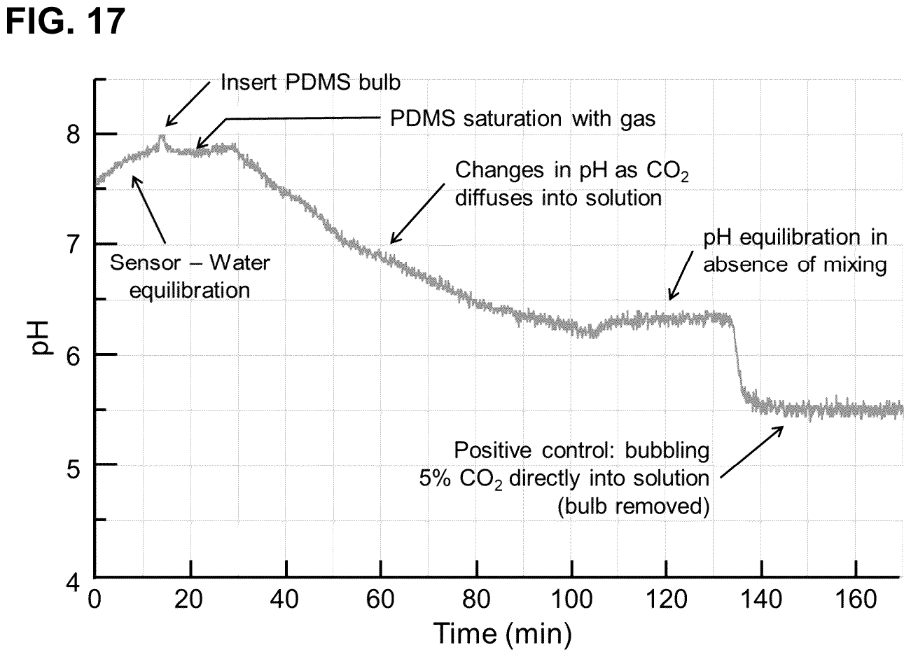

This invention offers the possibility of controlling the CO₂ concentration in both the headspace and the bulb, with a time constant set only by the gas exchange rate in the two spaces. Preliminary tests shown in FIG. 17 demonstrate that we can readily change pH by controlling the delivery of CO₂ to a DM-GEM bulb. In future embodiments, it would be possible to use a combination of proportional valves and time-division multiplexing of CO₂ to regulate pH independently in each of multiple wells.

### Other Embodiments

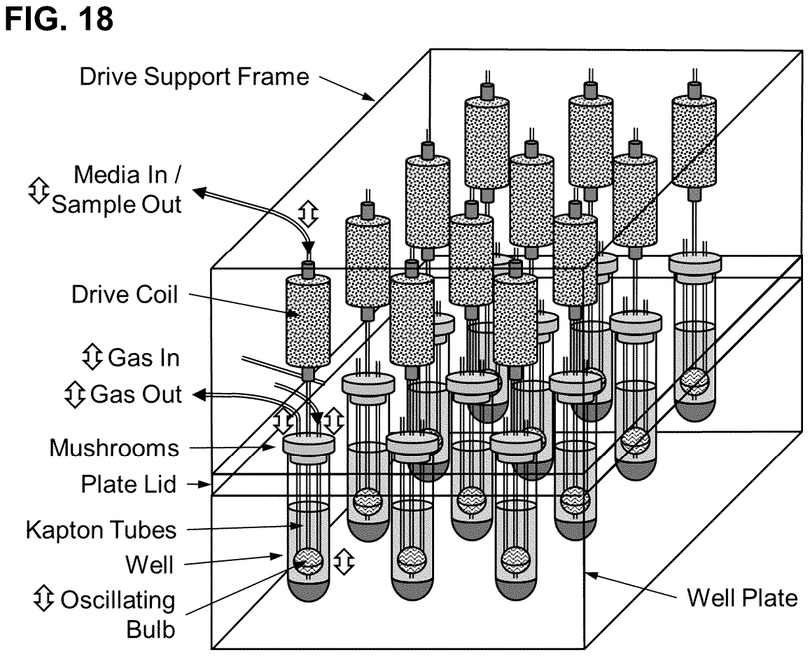

FIG. 18 presents an embodiment of the invention with twelve displacement membrane gas exchanger and mixer units operating in a well plate. The components that oscillate, including tubing, are marked with a double vertical arrow, and the Kapton tubes that are connected to the bulb slide through appropriately sized holes in the “mushrooms” that seal the top of each well.

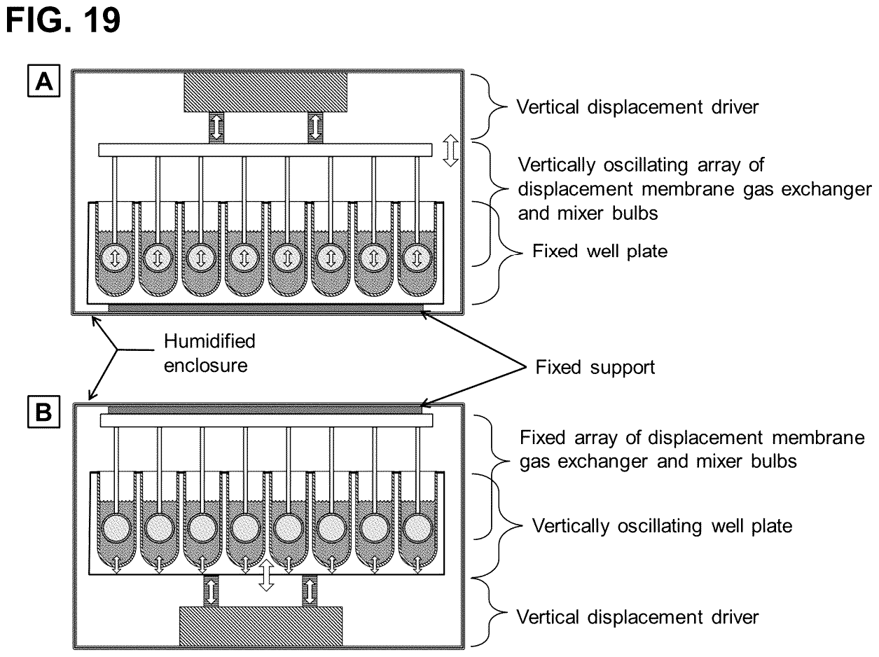

FIG. 19 presents alternative embodiments wherein an array of displacement membrane gas exchanger and mixer bulbs is moved relative to the well plate by either periodically raising and lowering the array (panel A of FIG. 19), or periodically raising and lowering the well plate (panel B of FIG. 19). The various tubes for delivery or removal of media and/or cells, not shown in this figure, can be attached to either the moving lid or the moving well plate. In the former case, the geometry of all tubes, sensors, and the displacement bulb are fixed and hence cannot limit the range of motion. In the latter case, the range of motion of either the displacement array or the well plate can be limited by interferences between the bulb and the fluid delivery and removal tubes and sensors. In contrast to the earlier designs, these two embodiments have the advantage of requiring only a single vertical actuator, while the disadvantage is that all displacers in the array must have the same amplitude and frequency of oscillation. One disadvantage of moving the plate rather than just the bulb array is the larger mass that must be accelerated and decelerated, and an increased risk of sloshing and cross-contamination of wells from sloshing. As drawn, the wells are open at the top and share a common humidified enclosure that also provide an aseptic environment to minimize microbial contamination. Alternatively, the drive tubes for each displacer can pass through small holes in either a common lid or individual “mushrooms”; the possibility of microbial contamination as the tubes slide through these holes would be minimized by placing the entire system inside the sterilized, humidified enclosure.

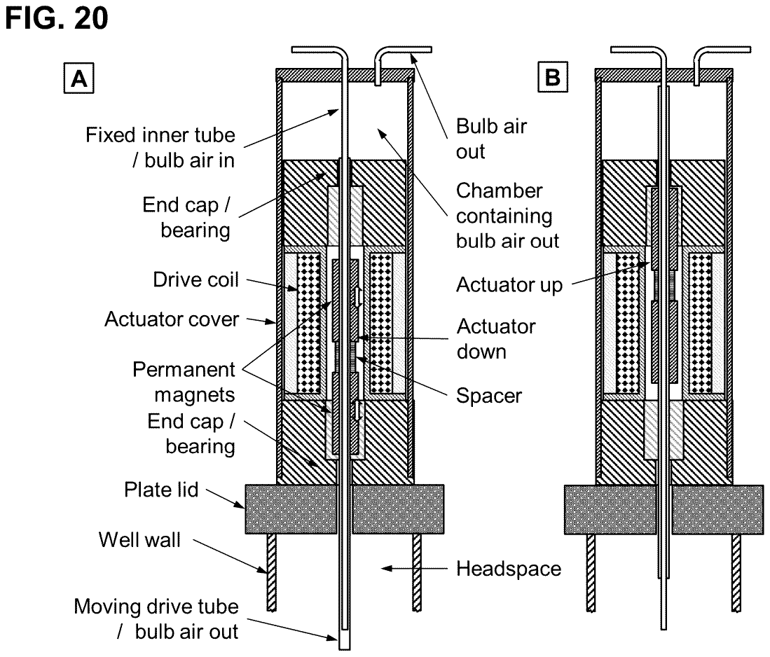

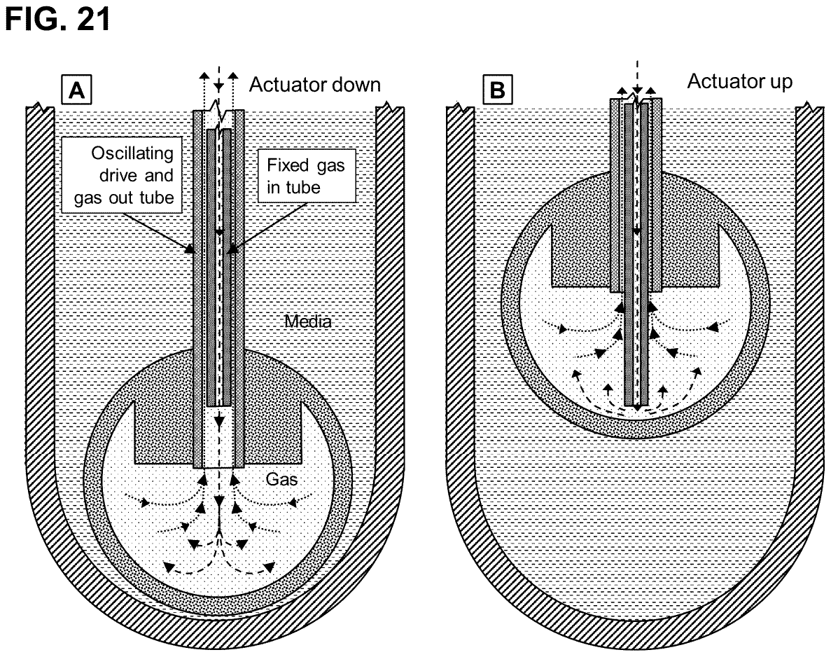

It would be useful to reduce the mechanical complexity of fabricating and driving a displacer with three tubes (gas in, gas out, and bottom media delivery and withdrawal as shown in FIGS. 7, 10, and 18) and also eliminate the requirement for flexible tubing connections to the moving components (the media in/sample out, gas in, and gas out tubes with double arrows in FIG. 18, for example) that complicates assembly and adds moving mass. The three exposed tubes above and sliding through the well plate lid in FIGS. 7 and 14 could introduce microbial contamination into the well. FIGS. 20 and 21 address these issues by implementing coaxial gas delivery and withdrawal from the bulb and eliminating the bottom-most media in/out tube. This design enables fabrication of bulbs with only a single cast port (FIG. 21), encloses all sliding tubes within a sterile space, and eliminates all moving interconnect tubing. As shown in FIGS. 20 and 21, there is a fixed inner gas delivery tube that is surrounded by a moving drive tube for bulb gas removal. The flexible tubing is no longer needed since the inner tube is fixed, and the outer tube terminates within the upper chamber of the actuator. While some mixing of incoming and outgoing gas will occur in the drive tube at the lowest ball positions, we expect that the small distances, high gas velocities in the small tube, and the short interval of time when the bulb is in its lowest position will minimize this problem.

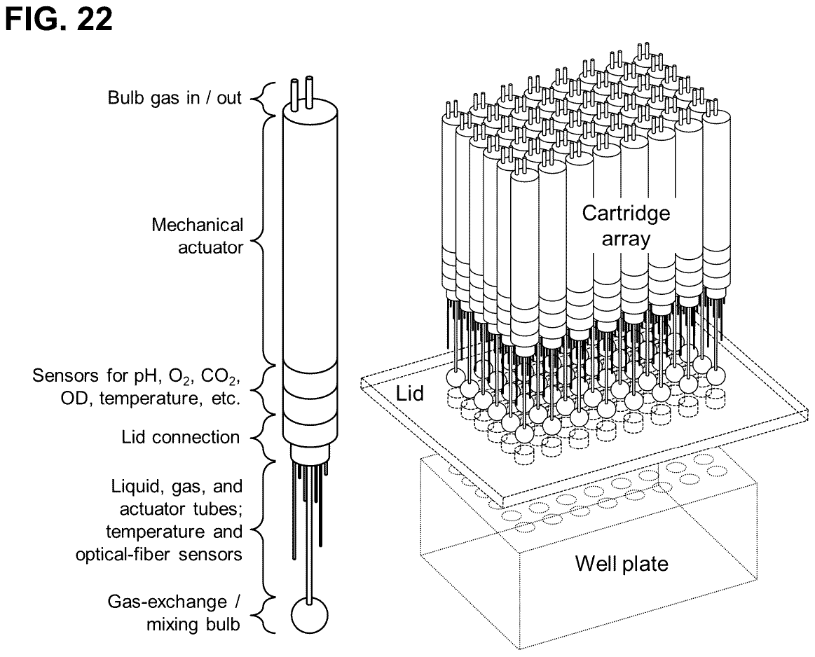

FIG. 22 shows how the concepts in FIGS. 20 and 21 can be merged to create a displacement membrane gas exchanger and mixer actuator that is fully compatible with self-contained cell culture instruments and is more readily produced in large quantities and serviced than the embodiments in FIGS. 7, 10, and 18. This cartridge could contain integrated sensors (either optical or electrochemical) for pH, temperature, O₂, CO₂, the optical density (OD) or electrical impedance of cell-containing media, cellular fluorescent reporters, lactate, ethanol, or other analytes of interest. Each of these cartridges can serve as a complete and independent displacement membrane gas exchanger and mixer for sensing and control of bioreactors that are implemented in the wells of a standard, commercial well plate. Any cartridge can be replaced individually, thereby simplifying servicing of the entire instrument.

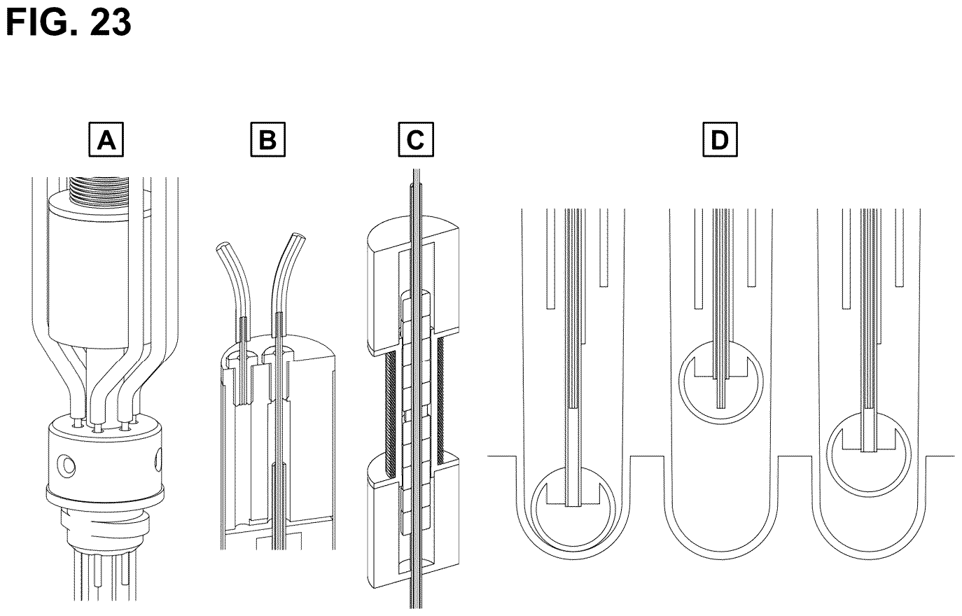

FIG. 23 provides close-up images of key aspects of the design. Panel A of FIG. 23 shows how the Tygon tubes and “mushroom” that are incorporated throughout our typical perfusion control systems can pass around the lower actuator end cap/bearing. Panel B of FIG. 23 illustrates how the fixed gas delivery tube that goes all the way down to the interior of the bulb is attached to the top of the actuator cover, while the gas exiting the drive tube coming up from the bulb can flow across and then up and out of the top of the actuator cover. Panel C of FIG. 23 shows how the two magnets that are attached to the drive tube are located within the solenoid and guided by the upper and lower bearings. Panel D of FIG. 23 illustrates how the inner gas delivery tube remains stationary while the bulb and the outer drive tube move up and down.

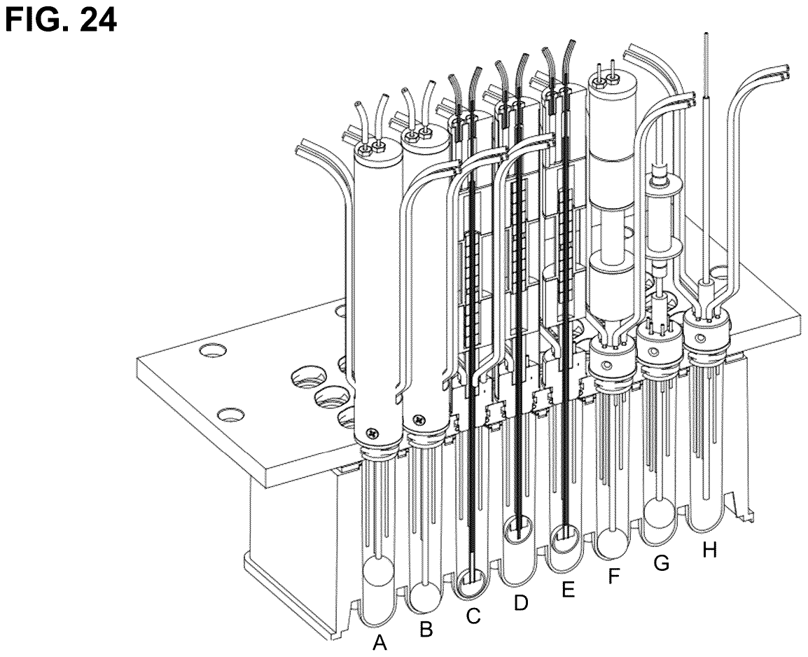

FIG. 24 shows eight actuators (A-H) in a single row of a deep 48-well plate. From left to right, actuators A and B illustrate displacement membrane gas exchanger and mixer bulbs in their upper and lower positions, while the cross-sections in actuators C, D, and E show the magnet positions within the solenoid in the low, high, and medium bulb positions. The outer cover is removed in actuator F. The bearings and end caps are removed in actuator G to show the magnets spanning the solenoid in the middle ball position. Actuator H illustrates the relation between the fixed inner gas delivery tube that protrudes above the coaxial, larger diameter but shorter gas removal tube, which is bonded to the magnets and is used to raise and lower the bulb. The concentric tube approach significantly simplifies the manufacture and servicing of displacement actuators and reduces the cost of implementing these complex components in a commercial instrument. Obviously, there are many possible refinements to and variations of the implementations in FIGS. 23 and 24, but the central theme is a single cartridge that can be inserted into one well of a multi-well plate to provide both mixing and gas exchange, with the possibility of including a variety of internal and in-line sensors.

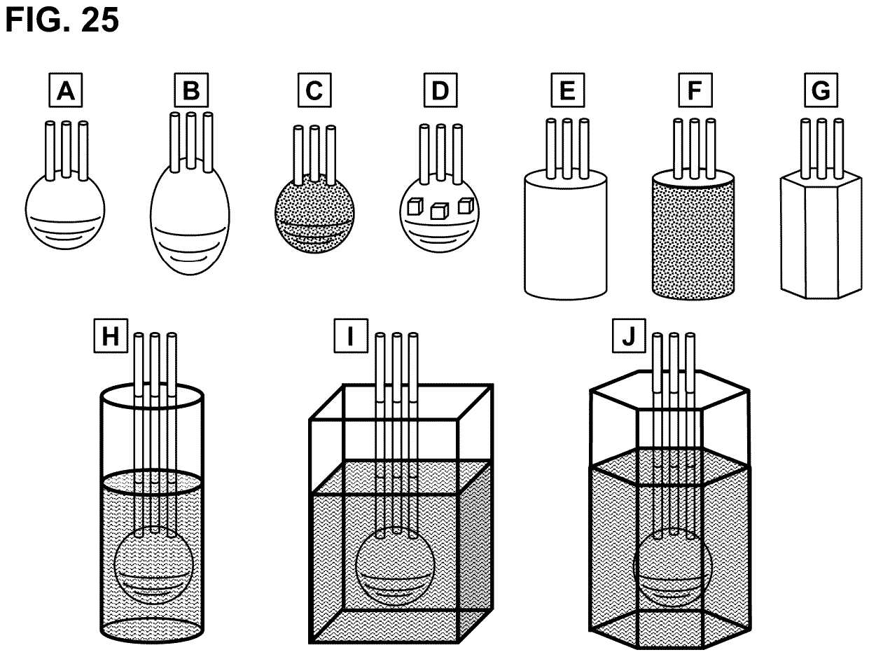

FIG. 25 illustrates a variety of configurations, surfaces, and shapes for displacement gas exchanger and mixer bulbs and the bioreactor vessels in which they operate. Panels A-B of FIG. 25 show spherical and spheroidal bulbs, panel C of FIG. 25 shows a bulb with a textured surface to increase both the surface area and interfacial mixing, and panel D of FIG. 25 has macroscopic surface features to enhance mixing and adjust local shear rates during bulb motion. Panels E-G of FIG. 25 are circular and hexagonal cylinders, for which there will be a longer laminar flow region between the bulb and the wall of a cylindrical bioreactor. These surfaces could be textured. All of the bulbs shown have three tubes - gas in, gas out, and bottom sampling. Other embodiments could use a single coaxial tube for gas delivery and exhaust as discussed previously for other system embodiments, and the bottom sampling tube need not be included. The bioreactors can be cylindrical with flat bottoms (panel H of FIG. 25), or have cross-sections that are rectangular (panel I of FIG. 25) or polygonal (panel J of FIG. 25).

This ability to move the bulb throughout much of the media in the bioreactor also provides a means of temperature control, since the displacement object makes close contact with the entire volume of the fluid within the bioreactor when the displacement bulb is constructed from a thermally conductive material. The temperature and flow rate of the gas into the displacement bulb can be regulated to control the fluid temperature, as could an electrical heater incorporated into the body of the displacer bulb. If the reactions within the bioreactor are exothermic, it would be possible to run the well plate enclosure and the delivered media slightly below the desired operating temperature so that the combination of the heat generated by the biochemical reactions within the bioreactor and the heat delivered by the displacement bulb would together be sufficient to support the desired operating temperature, with the rate of heat delivery by the displacement bulb serving as the variable used to support closed-loop temperature control.

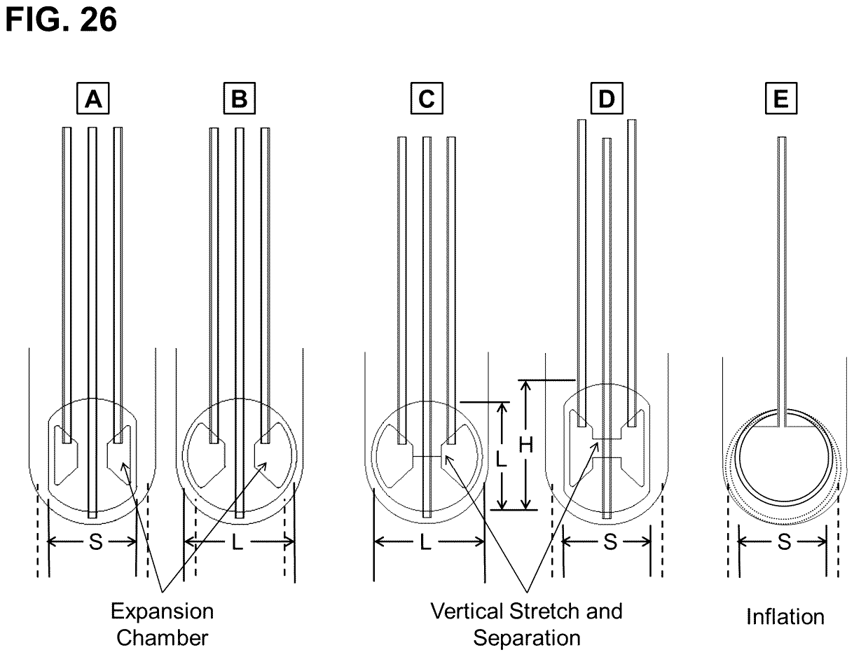

FIG. 26 shows three ways by which the diameter of the bulb could be adjusted dynamically to regulate gas exchange and mixing. In panels A-B of FIG. 26, adjustment of the pressure inside of an appropriately designed bulb could allow it to expand or contract to alter the width of the annular gap and hence the flow velocity past the gas-exchange membrane. Similarly, the vertical mechanical stretching of the bulb in panels D-C of FIG. 26 is accomplished in this example by the differential motion of the central tube attached to the lower half of the bulb and the gas delivery and removal tubes attached to the upper half. Finally, with the coaxial drive tube in panel E of FIG. 26, it is sufficient to regulate the operating pressure of the bulb to control its diameter, for example by using either a variable conductance valve on the output with a fixed input pressure, or a fixed output conductance and a variable input pressure, either possibly informed by a measurement of the pressure drop across the bulb.

With the exception of the occasional change in the diameter of the actuator as might be used to adjust the width of the annulus, none of the motions of the displacer affect the overall level of fluid in the bioreactor, which means that the height of the withdrawal tube above the bottom of the bioreactor vessel as shown in FIG. 1 controls the volume of the bioreactor, independent of the operation of the displacer as long as the displacer is never raised so high as to have its uppermost point break the surface of the fluid contained in the bioreactor vessel.

By maintaining a constant displacement volume of the mixing object, the fluid level of the vessel will remain the same throughout the stroke of the mixing object. This is a valuable feature for the operation of this invention, particularly when the system is operated in modes that require constant volume control. If the total volume of the reactor changes as a result of the mixing object changing volume, then there are only short periods in which fluid may be extracted based on the position of the fluid-gas interface and volume control becomes dependent upon both the frequency and amplitude of the stroke/volume change, which is also undesirable.

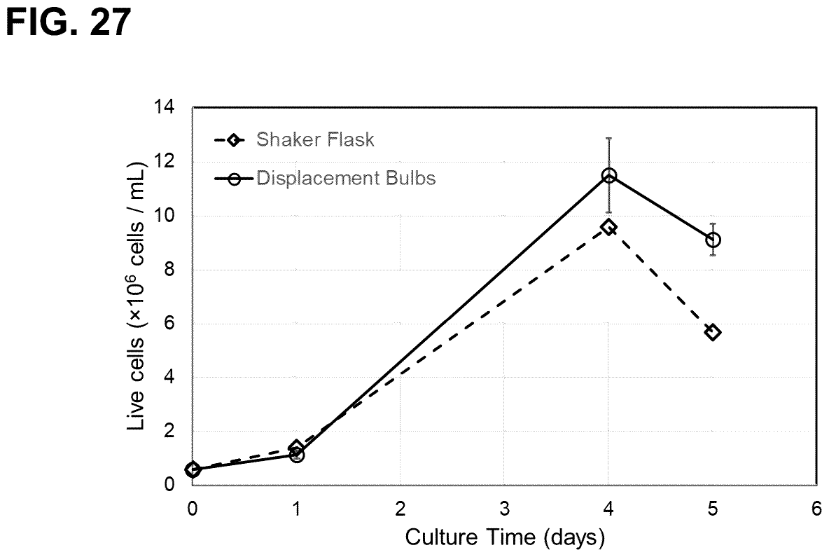

FIG. 27 is a demonstration that in batch-fed culture, a displacement membrane gas exchanger and mixer can grow CHO cells and achieve live cell densities comparable to or even better than a shaken flask, but that with both methods, the live cell density drops at Day 5 as nutrients are depleted and toxic metabolites accumulate. In practice, this could be addressed with a fed-batch reactor whose volume is increased during the course of the run, or by a perfusion reactor where media is removed and replaced on either a continuous or intermittent basis, with provision to minimize loss of cells from the reactor.

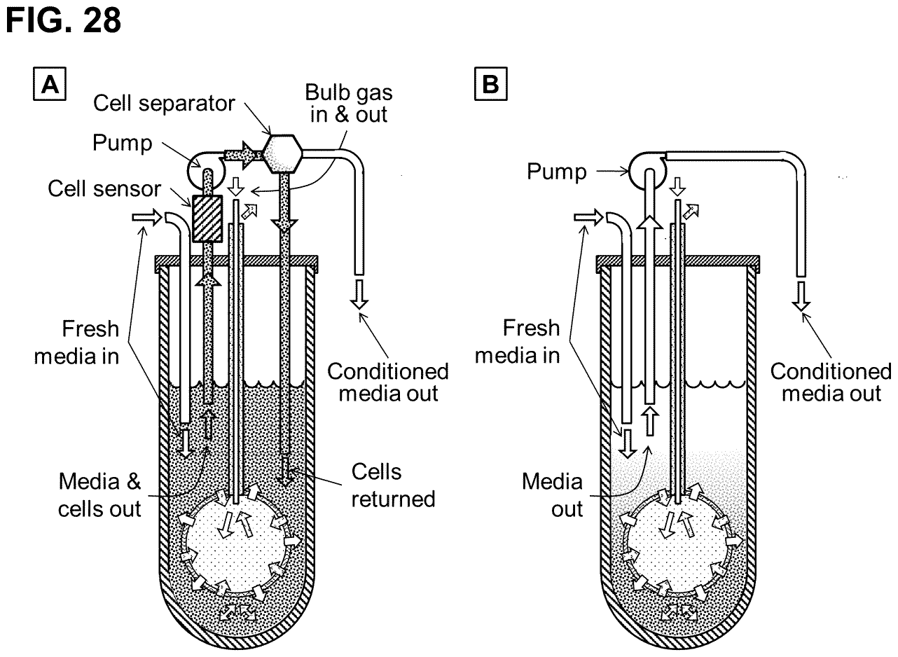

Panel A of FIG. 28 shows a bioreactor system that uses a displacement membrane gas exchanger and mixer, and either continuous or intermittent perfusion of media with the capture of suspended cells and their return to the bioreactor, with the cell separation and capture being accomplished with either tangential flow microfiltration, alternating tangential flow filtration, spiral or other kinetic cell separators, or other means. Note that the media and cell output line contains a cell sensor to determine either the cell density or the effluent optical density. Such a sensor could be added to any of the output lines in the other embodiments presented. Panel B of FIG. 28 shows another approach, used in some small bioreactors culturing suspended cells, to transiently halt mixing so as to allow the cells to settle such that cell-depleted media can be extracted from the bioreactor and replaced with fresh media prior to the resumption of mixing.

In sum, the invention offers, among other things, a number of significant advantages over other approaches for mixing and oxygenation. In stirring suspended cells with a rotating magnetic stir bar, the effectiveness of the stirring is determined by the shape and diameter of the bioreactor vessel, the length of the stir bar or impeller, the distance between the poles of the rotating bar magnet that drives the stirring, the stir speed, and the viscosity of the media being stirred. Higher stir bar or impeller speeds generally improve mixing speed and uniformity by increasing turbulence, but excessive velocity can lead to high shear forces, which may damage the cells or disrupt the cell suspension. The shear stress, quantified by the local spatial gradient of the fluid velocity, can directly affect cell viability. Low-shear impellers such as pitched-blade or marine impellers can be used for shear-sensitive cells. With a rotating stir bar, a longer stir bar creates a larger radius of rotation and increases the extent of the mixing zone. The velocity gradients, and hence the shear rate, will be greater at the end of the stir bar as compared to the middle, and greater for long bars than short ones. However, longer stir bars may improve the uniformity of the shear rate distribution across the bioreactor compared to a short stir bar. There are clear limitations in using a stir bar in a deep well, in that the stir bar length is limited by the well diameter, and with deep wells, a high stir speed is required to achieve adequate vertical mixing.

With regard to gas exchange, the cyclic movement of a large surface area over which gas can be exchanged offers many benefits over other techniques for oxygenating media and removing excess carbon dioxide. While headspace gas exchange may be straightforward in shallow wells, shaken well plates, or shaker flasks, it is clearly inadequate for deep wells containing high concentrations of metabolically active cells. Sparging of gas at the bottom of a deep bioreactor filled in protein-rich media can generate significant quantities of foam, which would disrupt cell culture unless defoaming agents are used, with the risk of disrupting the desired metabolic activity of the cells. The oscillating displacer membrane ensures that the gas transfer surfice is generally in contact with media that has not yet been fully oxygenated, hence optimizing both gas transfer and homogeneity of dissolved gases. Furthermore, if the displacer bulb is elastomeric, the internal gas pressure also controls the bulb's diameter, allowing mixing rates to be tuned dynamically as the medium viscosity increases with rising cell density.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of the invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference were individually incorporated by reference.

### REFERENCES

[1] R. Zheng, N. Phanthien, R. I. Tanner, The flow past a sphere in a cylindrical tube-effects of inertia, shear-thinning and elasticity, Rheol. Acta 30(6) (1991) 499-510.
[2] S. Taneda, Experimental investigation of the wake behind a sphere at low Reynolds numbers, J. Phys. Soc. Jpn. 11(10) (1956) 1104-1108.
[3] F. Garcia-Ochoa, E. Gomez, Bioreactor scale-up and oxygen transfer rate in microbial processes: An overview, Biotechnol. Adv. 27(2) (2009) 153-176.

What is claimed is:

1. A bioreactor system, comprising:
a bioreactor vessel configured to contain a culture medium; and
a displacement membrane gas exchanger and mixer (DM-GEM) configured to be positioned within the vessel, wherein the DM-GEM comprises:
a displacement member having an outer cross-sectional area smaller than an inner cross-sectional area of the vessel, thereby defining a fluid annulus between the displacement member and a wall of the vessel, wherein the displacement member has a gas-permeable surface defining an interior chamber therewith; and
an actuator mechanism coupled to the displacement member and configured to vertically oscillate the displacement member through a substantial portion of the culture medium in the vessel,
wherein vertical oscillation of the displacement member displaces the culture medium through the fluid annulus between the displacement member and the vessel wall to mix the medium while simultaneously exchanging gas across the gas-permeable wall of the displacement member.

2. The system of claim 1, wherein the gas-permeable surface of the displacement member is configured to permit diffusion of oxygen from the interior chamber into the culture medium and diffusion of carbon dioxide from the culture medium into the interior chamber.

3. The system of claim 1, wherein the displacement member is an extensible bulb formed of an elastomer, and a diameter of the extensible bulb varies in response to gas pressure within the interior chamber.

4. The system of claim 1, wherein the vertical oscillation of the displacement member is configured to:
force the culture medium to flow through the annulus between the displacement member and the vessel wall;
generate laminar flow within the annulus to minimize shear forces on suspended cells; and
generate vortices and turbulent mixing in the wake region above and below the displacement member to promote large-scale mixing throughout the culture medium.

5. The system of claim 1, wherein the actuator mechanism is an electromagnetic actuator comprising a solenoid and at least one permanent magnet coupled to the displacement member, wherein current in the solenoid is controlled to generate the vertical oscillation of the displacement member.

6. The system of claim 1, wherein gas pressure within the interior chamber is controlled to regulate at least one of a gas transfer rate, a mixing intensity, and a shear rate experienced by cells.

7. The system of claim 1, wherein the displacement member is oscillated with a waveform selected to control a shear rate and wake vortex generation within the culture medium.

8. The system of claim 1, further comprising a gas management unit configured to:
deliver a gas mixture into the culture medium via diffusion and/or convection across the gas-permeable wall of the displacement member;
receive metabolic gases from the culture medium via diffusion into the interior chamber of the displacement member; and/or
dynamically alter dimensions of the displacement member to adjust a width of the fluid annulus and tune a mixing rate.

9. The system of claim 1, further comprising:
a gas supply fluidly coupled to the interior chamber of the displacement member to deliver the gas mixture with a controlled gas composition;
a headspace coupled to the vessel and configured to control gas partial pressures and flow rates in a headspace of the bioreactor vessel for additional gas exchange; and
a humidification unit to humidify gas delivered to both the displacement member and the headspace to prevent evaporation of the culture medium.

10. The system of claim 1, wherein the DM-GEM further functions for temperature control, by regulating the temperature and flow rate of the gas delivered to the interior chamber of the displacement member to control the temperature of the culture medium.

11. The system of claim 1, wherein the DM-GEM is implemented as a cartridge configured for insertion into a well of a multi-well plate, wherein the cartridge further comprises coaxial conduits including a fixed inner tube delivering gas to the interior chamber and a movable outer tube removing gas from the interior chamber and controlling the vertical position of the displacement member.

12. The system of claim 1, wherein the bioreactor vessel is one well of a multi-well plate, and the system further comprises a plurality of displacement members operating in parallel in respective wells.

13. The system of claim 1, further comprising one or more sensors configured to measure at least one of pH, temperature, dissolved oxygen, carbon dioxide, lactate, optical density, and metabolite concentration in the culture medium.

14. The system of claim 1, further configured for perfusion operation, including a means for continuous or intermittent removal of spent media and replacement with fresh media while retaining suspended cells in the bioreactor vessel.

15. A bioreactor system, comprising:
a bioreactor vessel configured to contain a culture medium;
a gas-permeable member dimensioned to be moved vertically within the vessel to define a fluid annulus between the gas-permeable member and a wall of the vessel; and
a drive mechanism operably coupled to the gas-permeable member, and configured to cycle a vertical position of the gas-permeable member to displace the culture medium and promote mixing while simultaneously facilitating gas diffusion and/or convection across a surface of the gas-permeable member.

16. The system of claim 10, wherein the gas-permeable member is an extensible bulb.

17. The system of claim 10, further comprising a means for adjusting the internal pressure of the gas-permeable member to modify dimensions of the gas-permeable member, thereby controlling a width of the annulus and dynamically tuning a rate of displacement mixing.

18. The system of claim 10, wherein the vertical movement of the gas-permeable member is configured to generate:
laminar fluid flow through the annulus to minimize shear forces on cultured cells in the culture medium; and/or
turbulent fluid flow (vortices) in bulk culture medium upstream and downstream of the gas-permeable member to promote homogenization.

19. The system of claim 10, further comprising a gas management system configured to flow a gas mixture through an interior of the gas-permeable member to enable bidirectional gas exchange, including delivery of oxygen and removal of carbon dioxide.

20. The system of claim 10, wherein the drive mechanism comprises a mechanical crank, a pneumatic piston, a pneumatic bellows, and/or an electromagnetic actuator.
